(19) United States
(12) Patent Application Publication
(10) Pub. No.: US 2023/0406808 A1
(43) Pub. Date: Dec. 21, 2023

(54) DEHYDRATION OF LACTIC ACID AND RELATED COMPOUNDS IN SOLID ACIDS VIA MULTIFUNCTIONAL FLEXIBLE MODIFIERS

(71) Applicant: Regents of the University of Minnesota, Minneapolis, MN (US)

(72) Inventors: Paul Jakob Dauenhauer, Minneapolis, MN (US); Yutong Pang, Minneapolis, MN (US)

(21) Appl. No.: 18/037,179

(22) PCT Filed: Nov. 17, 2021
(86) PCT No.: PCT/US2021/059765

§ 371 Date: May 16, 2023

Related U.S. Application Data
(60) Provisional application No. 63/114,758, filed on Nov. 17, 2020.

(51) Int. Cl. C07C 67/327; B01J 29/08

(52) U.S. Cl. CPC C07C 67/327; B01J 29/084; B01J 2229/37

(57) ABSTRACT

A catalyst composition includes a solid acid catalyst having a multiplicity of acid sites on the surfaces and a multifunctional component coupled to the surfaces of the solid acid catalyst. Each multifunctional component includes at least two functional groups configured to accept a proton from an acid site of the multiplicity of acid sites. The catalyst composition can be used to dehydrate lactic acid, a lactic acid ester, a lactic acid salt, or a combination thereof, to yield a product comprising acrylic acid, an acrylic acid ester, an acrylic acid salt, or a combination thereof.

### CROSS-REFERENCE TO RELATED APPLICATION

This application claims the benefit of U.S. Patent Application No. 63/114,758 filed on Nov. 17, 2020, which is incorporated by reference herein in its entirety.

### TECHNICAL FIELD

This invention relates to dehydration of lactic acid, lactic acid esters, and lactic acid salts in solid acid catalysts via multifunctional flexible modifiers.

### BACKGROUND

The manufacturing of acrylic acid and its esters is important for the preparation of polyacrylic acid polymers and other monomers including acrylamides, acrylonitrile, and other vinyl compounds. These materials can then be used in the manufacture of various plastics, coatings, adhesives, elastomers, polishes, and paints. While acrylic acid is produced from the oxidation of propylene, there exist new methods to synthesize it from biomass-derived resources. For example, fermentation of glucose can produce the chemical building block 3-hydroxy-propionic acid, which can be dehydrated to acrylic acid. Alternatively, another chemical building block of lactic acid (2-hydroxypropanoic acid) produced from glucose fermentation can also be dehydrated to acrylic acid using acid catalysts. FIG. 1 depicts dehydration of lactate or lactic acid to acrylate or acrylic acid, where X is a cation (e.g., hydrogen, ammonium, lithium, sodium, potassium, cesium, magnesium, calcium, strontium, or barium) or an alkyl or cycloalkyl group with 1 to 10 carbon atoms, methyl, ethyl, n-propyl, isopropyl, n-butyl, s-butyl, isobutyl, n-pentyl, isopentyl, cyclopentyl, n-hexyl, cyclohexyl, n-heptyl, methylcyclohexyl, n-octyl, 2-ethylhexyl, bornyl, or isobornyl).

### SUMMARY

This disclosure describes dehydration of lactic acid, lactic acid esters, and lactic acid salts on solid acids via multifunctional flexible modifiers to improve selectivity for acrylic acid, acrylic acid esters, or salts of acrylic acid. Examples of solid acids include zeolites, such as FAU zeotype materials including Na-FAU. Dehydration selectivity of over 90 C-mol % has been achieved. In one example, 1,2-bis(4-pyridyl-ethane) exhibits selectivity in excess of 97% to acrylics over a modified Na-FAU zeolite catalyst. As described herein, multifunctional flexible amines are surprisingly effective despite being relatively weak bases.

Although the disclosed inventive concepts include those defined in the attached claims, it should be understood that the inventive concepts can also be defined in accordance with the following embodiments.

In addition to the embodiments of the attached claims and the embodiments described above, the following numbered embodiments are also innovative.

Embodiment 1 is a composition comprising:
a solid acid catalyst comprising a multiplicity of acid sites on the surfaces; and
a multifunctional component coupled to the surfaces of the solid acid catalyst, wherein each multifunctional component comprises at least two functional groups configured to accept a proton from an acid site of the multiplicity of acid sites.

Embodiment 2 is the composition of embodiment 1, further comprising a cation comprising hydrogen, ammonium, lithium, sodium, potassium, cesium, magnesium, calcium, strontium, barium, or a combination thereof.

Embodiment 3 is the composition of embodiment 1 or 2, wherein the solid acid catalyst comprises a zeolite.

Embodiment 4 is the composition of embodiment 3, wherein the zeolite comprises 12-ring pore openings.

Embodiment 5 is the composition of embodiment 3, wherein the zeolite comprises a FAU zeotype.

Embodiment 6 is the composition of any one of embodiments 1 through 5, wherein the solid acid catalyst comprises one or more of FAU (Y), silicalite-1 (MFI), zeolite beta (BEA), mordenite (MOR), and SAO.

Embodiment 7 is the composition embodiment 1, wherein the solid acid catalyst comprises one or more of silica-alumina, MCM-41, silica gel, metal organic frameworks, and covalent organic frameworks.

Embodiment 8 is the composition of embodiment 7, wherein the solid acid catalyst is a metal organic framework, and the metal organic framework is a zeolitic imidazolate framework.

Embodiment 9 is the composition of any one of embodiments 1 through 8, wherein the solid acid catalyst comprises aluminum.

Embodiment 10 is the composition of embodiment 9, wherein the solid acid catalyst comprises at least 0.00001 wt % aluminum.

Embodiment 11 is the composition of any one of embodiments 1 through 10, wherein the solid acid catalyst comprises one or more of Na, Li, K, Rb, Cs, Cu, Fe, Co, La, Ce, Sm, Eu, Ag, Mg, Ca, Sr, and Ba.

Embodiment 12 is the composition of any one of embodiments 1 through 11, wherein at least one of the at least two functional groups is an amine functional group.

Embodiment 13 is the composition of embodiment 12, wherein at least two of the at least two functional groups are amine functional groups.

Embodiment 14 is the composition of any one of embodiments 1 through 13, wherein the multifunctional component comprises one of the Class I compounds depicted in FIG. 14, wherein each of R₁-R₅ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 15 is the composition of any one of embodiments 1 through 13, wherein the multifunctional component comprises one of the Class II and Class III compounds depicted in FIG. 15, wherein each of R₆-R₁₀ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 16 is the composition of any one of embodiments 1 through 13, wherein the multifunctional component comprises one of the Class IV compounds depicted in FIG. 16, wherein each R independently represents H, methyl, ethyl, propyl, or isopropyl, and R₁₁ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 17 is the composition of any one of embodiments 1 through 13, wherein the multifunctional component comprises one of the Class V and Class VI compounds depicted in FIG. 17, wherein each R independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 18 is the composition of any one of embodiments 1 through 13, wherein the multifunctional component comprises one of the compounds depicted in FIG. 18.

Embodiment 19 is the composition of any one of embodiments 1 through 18, wherein, using a baseline of a normal axis of a first one of the at least two functional groups, a geometric flexibility angle of a multifunctional component is the angle or set of angles accessible by a second one of the at least two functional groups, and the geometric flexibility angle is greater than 5 degrees.

Embodiment 20 is the composition of embodiment 19, wherein the multifunctional component comprises a minimum geometric flexibility angle and a maximum geometric flexibility angle, and a difference between the minimum geometric flexibility angle and the maximum geometric flexibility angle is greater than 5 degrees.

Embodiment 21 is the composition of any one of embodiments 1 through 20, wherein the at least two functional groups are separated by a spacer.

Embodiment 22 is the composition of embodiment 21, wherein the spacer comprises an alkyl group, a heteroalkyl group, an alkenyl group, a heteroalkenyl group, a cycloalkyl group, a heterocyclyl group, an aryl group, or a heteroaryl group.

Embodiment 23 is the composition of embodiment 22, wherein the alkyl group, the heteroalkyl group, the alkenyl group, or the heteroalkenyl group comprises 1-20 carbon atoms.

Embodiment 24 is the composition of any one of embodiments 22 or 23, wherein the heteroalkyl group, the heteroalkenyl group, the heterocyclyl group, or the heteroaryl group comprises one or more of S, Cl, Br, B, F, Si, P, N, and O.

Embodiment 25 is method of dehydrating a reactant comprising lactic acid, a lactic acid ester, a lactic acid salt, or a combination thereof, the method comprising:
contacting a solid acid catalyst with the reactant, wherein
the solid acid catalyst comprises:
   surfaces defining pores; and
   a multiplicity of acid sites on the surfaces,
a multifunctional component is coupled to the surfaces of the solid acid catalyst, wherein each multifunctional component comprises at least two functional groups, and each functional group is configured to accept a proton from an acid site of the multiplicity of acid sites; and
dehydrating the reactant to yield a product comprising acrylic acid, an acrylic acid ester, an acrylic acid salt, or a combination thereof.

Embodiment 26 is the method of embodiment 25, wherein the solid acid catalyst comprises a zeolite.

Embodiment 27 is the method of embodiment 26, wherein the solid acid catalyst comprises one or more of FAU (Y), silicalite-1 (MFI), zeolite beta (BEA), mordenite (MOR), and SAO.

Embodiment 28 is the method of embodiments 26 or 27, wherein the solid acid catalyst comprises one or more of silica-alumina, MCM-41, silica gel, metal organic frameworks, and covalent organic frameworks.

Embodiment 29 is the method of embodiment 26, wherein the solid acid catalyst is a metal organic framework, and the metal organic framework is a zeolitic imidazolate framework.

Embodiment 30 is the method of any one of embodiments 25-29, wherein the solid acid catalyst comprises aluminum.

Embodiment 31 is the method of embodiment 30, wherein the solid acid catalyst comprises at least 0.00001 wt % aluminum.

Embodiment 32 is the method of any one of embodiments 25 through 31, wherein the solid acid catalyst comprises one or more of Na, Li, K, Rb, Cs, Cu, Fe, Co, La, Ce, Sm, Eu, Ag, Mg, Ca, Sr, and Ba.

Embodiment 33 is the method of any one of embodiments 25 through 32, wherein at least one of the at least two functional groups is an amine functional group.

Embodiment 34 is the method of embodiment 33, wherein at least two of the at least two functional groups are amine functional groups.

Embodiment 35 is the method of any one of embodiments 25 through 34, wherein the multifunctional component comprises one of the Class I compounds depicted in FIG. 14, wherein each of R₁-R₅ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 36 is the method of any one of embodiments 25 through 34, wherein the multifunctional component comprises one of the Class II and Class III compounds depicted in FIG. 15, wherein each of R₆-R₁₀ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 37 is the method of any one of embodiments 25 through 34, wherein the multifunctional component comprises one of the Class IV compounds depicted in FIG. 16, wherein each R independently represents H, methyl, ethyl, propyl, or isopropyl, and R₁₁ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 38 is the method of any one of embodiments 25 through 34, wherein the multifunctional component comprises one of the Class V and Class VI compounds depicted in FIG. 17, wherein each R independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

Embodiment 39 is the method of any one of embodiments 25 through 34, wherein the multifunctional component comprises one of the compounds depicted in FIG. 18.

Embodiment 40 is the method of any one of embodiments 25 through 39, wherein, using a baseline of a normal axis of a first one of the at least two functional groups, a geometric flexibility angle of a multifunctional component is the angle or set of angles accessible by a second one of the at least two functional groups, and the geometric flexibility angle is greater than 5 degrees.

Embodiment 41 is the method of embodiment 40, wherein the multifunctional component comprises a minimum geometric flexibility angle and a maximum geometric flexibility angle, and a difference between the minimum geometric flexibility angle and the maximum geometric flexibility angle is greater than 5 degrees.

Embodiment 42 is the method of any one of embodiments 25 through 41, wherein the at least two functional groups are separated by a spacer.

Embodiment 43 is the method of embodiment 42, wherein the spacer comprises an alkyl group, a heteroalkyl group, an alkenyl group, a heteroalkenyl group, a cycloalkyl group, a heterocyclyl group, an aryl group, or a heteroaryl group.

Embodiment 44 is the method of embodiment 43, wherein the alkyl group, the heteroalkyl group, the alkenyl group, or the heteroalkenyl group comprises 1-20 carbon atoms.

Embodiment 45 is the method of embodiments 43 or 44, wherein heteroalkyl group, the heteroalkenyl group, the heterocyclyl group, or the heteroaryl group comprises one or more of S, Cl, Br, B, F, Si, P, N, and O.

Embodiment 46 is the method of any one of embodiments 25 through 45, wherein the selectivity of the dehydrating is at least 80 C-mol %.

Embodiment 47 is the method of embodiment 46, wherein the selectivity of the dehydrating is at least 85 C-mol %.

Embodiment 48 is the method of embodiment 47, wherein a selectivity of the dehydrating is at least 90 C-mol %.

Embodiment 49 is the method of any one of embodiments 25 through 48, further comprising providing a fluid comprising the multifunctional component to a vessel containing the solid acid catalyst.

Embodiment 50 is the method of embodiment 49, wherein the fluid comprises a gas or a liquid.

Embodiment 51 is the method of embodiment 50, wherein the fluid comprises a liquid, and further comprising vaporizing the liquid.

Embodiment 52 is the method of any one of embodiments 25 through 48, wherein contacting the solid acid catalyst with the reactant comprises providing a fluid comprising a solvent and the reactant to a vessel containing the solid acid catalyst.

Embodiment 53 is the method of any one of embodiments 25 through 48, wherein contacting the solid acid catalyst with the reactant comprises providing a fluid comprising a solvent and reactant to a vessel containing the solid acid catalyst.

Embodiment 54 is the method of embodiment 53, wherein the solvent comprises one or more of water, an alcohol, and an ester.

Embodiment 55 is the method of embodiment 54, wherein a concentration of the reactant is at least 0.0001 wt % and less than 100 wt %.

Embodiment 56 is the method of any one of embodiments 53 through 55, further comprising vaporizing the fluid.

Embodiment 57 is the method of embodiment 56, wherein a partial pressure of the reactant is in a range between about 0.01 Pa and about 5000 Pa.

Embodiment 58 is the method of any one of embodiments 25 through 57, wherein dehydrating the reactant occurs at a reaction temperature between about 50° C. and about 500° C.

Embodiment 59 is the method of any one of embodiments 25 through 58, wherein dehydrating the reactant occurs in a vessel, and a space velocity of the reactant is in a range between 0.001 and 100 h⁻¹.

Embodiment 60 is the method of any one of embodiments 25 through 59, wherein a molar ratio of the multifunctional component to the reactant is in a range between about 0.001 to about 1000.

Embodiment 61 is the method of any one of embodiments 25 through, 60wherein a weight ratio of the multifunctional component to the solid acid catalyst during reaction is in a range between about 0.001 wt % and about 100 wt %.

Embodiment 62 is a composition comprising:
a solid acid comprising a multiplicity of acid sites on the surfaces;
a multifunctional component comprising at least two functional groups, each functional group configured to accept a proton from one of the acid sites; and
a reactant comprising lactic acid, a lactic acid ester, a lactic acid salt, or a combination thereof.

Embodiment 63 is the composition of embodiment 62, further comprising a cation comprising hydrogen, ammonium, lithium, sodium, potassium, cesium, magnesium, calcium, strontium, barium, or a combination thereof.

Embodiment 64 is the composition of embodiment 62 or 63, wherein the solid acid comprises a zeolite.

Embodiment 65 is the composition of embodiment 64, wherein the zeolite comprises 12-ring pore openings.

Embodiment 66 is the composition of any one of embodiments 62 through 65, wherein the zeolite comprises a FAU zeotype.

Embodiment 67 is the composition of any one of embodiments 62 through 66, wherein the acid sites are Bronsted acid sites, ion-exchanged acid sites, or a combination thereof.

Embodiment 68 is the composition of any one of embodiments 62 through 67, further comprising acrylic acid, acrylic acid ester, or acrylic acid salt.

Embodiment 69 is the composition of any one of embodiments 62 through 68, wherein a temperature of the reaction mixture is in a range between about 50° C. and about 500° C.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 depicts dehydration of lactic acid.

FIG. 2 shows proton affinity over the non-dimensionalized size of amines. The non-dimensionalized size is calculated by normalizing the van der Waals diameter of the amine by the pore diameter of Na-FAU. The van der Waals diameter of the amine is calculated with QSAR Toolbox, and the pore diameter of Na-FAU is measured to be 7.17 Å by Argon physisorption at 87 K.

FIG. 3 shows acetaldehyde productivity as a function of time-on-stream (TOS) with the addition of amines. Deactivation constant (4) with different amines are shown in the bar chart. Reaction conditions: 300° C., Pₜₒₜₐₗ=101.3 kPa (P_{methyl lactate}=78.0 Pa, Pₐₘᵢₙₑ=7.8 Pa, P_{water}=1.1 kPa, balanced by He), WHSV=0.9 h⁻¹.

FIG. 4 shows dehydration selectivity of methyl lactate over Na-FAU with the addition of amines of various proton affinity and size.

FIG. 5 depicts three proposed steps in the in-situ Bronsted acid sites titration by amines: from left to right: amine transport to Bronsted acid sites via internal diffusion; competitive adsorption against methyl lactate; and desorption from Bronsted acid site.

FIG. 6 shows methyl lactate dehydration selectivity under pre-saturated conditions (black) in comparison with the value from co-feed conditions (diagonal bars) with the addition of 2,6DEP, 2,6DIPP, and 2,6DTBP. Reactivity measurements were performed at 300° C., Pₜₒₜₐₗ=101.3 kPa (P_{methyl lactate}=78.0 Pa, Pₐₘᵢₙₑ=7.8 Pa, P_{water}=1.1 kPa, balanced by He), and WHSV=0.9 h⁻¹.

FIG. 7 shows that a potential dehydration selectivity as high as 90% may be achieved with optimal bases.

FIG. 8A shows dehydration selectivity of methyl lactate over Na-FAU with the addition of predicted amines with 1,1,3,3-tetramethylguanidine (TMG) in filled squares, N, N, N′, N′-tetramethylethylenediamine in unfilled triangles with a dot in the center, and 1,2-dimethylimidazole in unfilled circles. FIG. 8B shows reactivities of methyl lactate over Na-FAU with 1,1,3,3-tetramethylguanidine (TMG), with carbon balance in asterisks, dehydration selectivity in filled squares, and conversion in crosses, at reaction conditions of 300° C., Pₜₒₜₐₗ=101.3 kPa (P_{methyl lactate}=78.0 Pa, Pₐₘᵢₙₑ=7.8 Pa, P_{water}=1.1 kPa, balanced by He), and WHSV=0.9 h⁻¹.

FIGS. 9A and 9B show dehydration selectivity and conversion of methyl lactate, respectively, over Na-FAU with the addition of 1,2-bis(4-pyridyl)ethane (1,2BPE) under continuous flow (in filled squares) and paused flow (in unfilled squares) conditions. FIG. 9C shows a repeat of the paused flow experiment with 1,2BPE to capture the reproducibility of using diamine under the same reaction condition to enhance methyl lactate dehydration selectivity above 90 C-mol %. Selectivity values from the same experiment as shown in FIG. 9A are shown in open circles and values from the repeated experiment under the same condition are shown in asterisks. FIGS. 9D and 9E show dehydration selectivity and conversion of methyl lactate, respectively, over Na-FAU plotted with 4,4′-trimethylenedipyridine under continuous flow (in filled circles), paused flow at 40 min TOS (in unfilled circles), and paused flow at 120 min TOS (in unfilled squares with cross) condition. Reaction conditions: 300° C., Pₜₒₜₐₗ=101.3 kPa (P_{methyl lactate}=78.0 Pa, P_{diamine}=3.9 Pa, P_{water}=1.1 kPa, balanced by He), and WHSV=0.9 h⁻¹.

FIGS. 10A-10D show the dehydration selectivity and conversion of methyl lactate over diamine loaded Na-FAU samples with varying loadings from 1 to 40 wt %. Data with 4,4′TMDP (4,4′-trimethylenedipyridine) are shown in FIGS. 10A and 10B. Data with 1,2BPE (1,2-bis(4-pyridyl)ethane) are shown in FIGS. 10C and 10D. Data collected with no pre-treatment step are presented in filled symbols and data with pre-treatment step are presented in unfilled symbols. Reaction conditions: 300° C., Pₜₒₜₐₗ=101.3 kPa (P_{methyl lactate}=78.0 Pa, P_{water}=1.1 kPa, balanced by He), and WHSV=0.9 h⁻¹.

FIG. 11 shows dehydration selectivity of methyl lactate over Na-FAU under continuous flow conditions with the addition of diamines plotted over the normalized size of diamines. The normalized size is calculated by the ratio between the maximum diamine diameter and Na-FAU supercage diameter. Reaction condition: Pₜₒₜₐₗ=101.3 kPa (P_{methyl lactate}=78 Pa, P_{nitrogen}=7.8 Pa, P_{water}=1.1 kPa, balanced by He), and WHSV=0.9 h⁻¹. The labels from left to right are provided in Table 5.

FIGS. 12A and 12B depict the geometric flexibility of the multifunctional component (here, diamines) as defined by the minimum and maximum geometric angles and distances that can be formed between the two nitrogen atoms.

FIG. 13A shows a plot of methyl lactate conversion against time with diamines under continuous flow condition. FIG. 13B shows the deactivation constant (k) for diamines in FIG. 13A.

FIG. 14 shows examples of Class I multifunctional components.

FIG. 15 shows examples of Class II and Class III multifunctional components.

FIG. 16 shows examples of Class IV multifunctional components.

FIG. 17 shows examples of Class V and Class VI multifunctional components.

FIG. 18 shows examples of heteroatom multifunctional components.

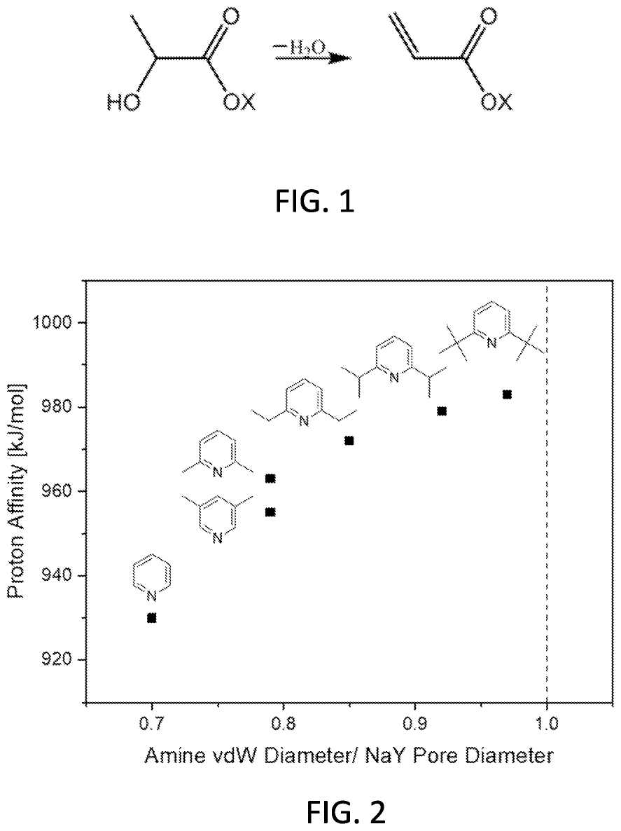

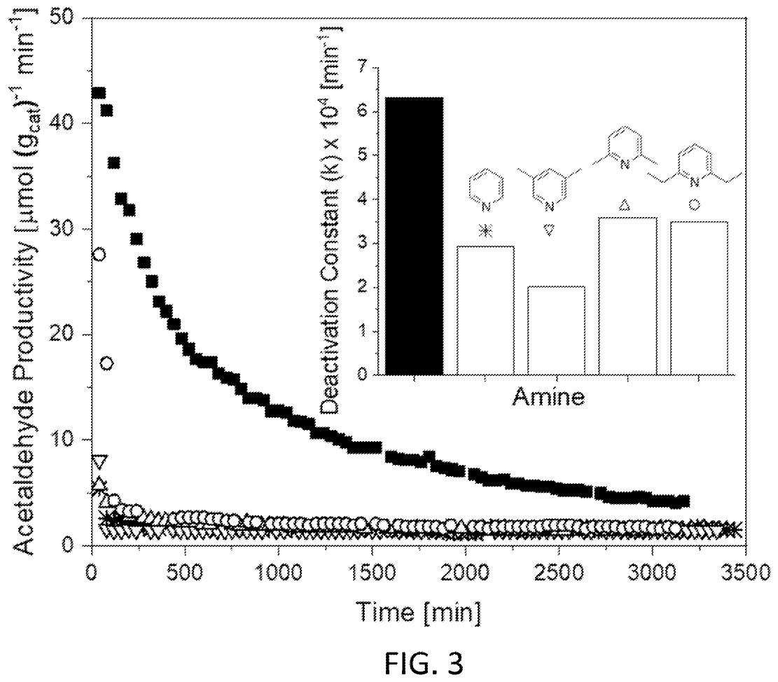

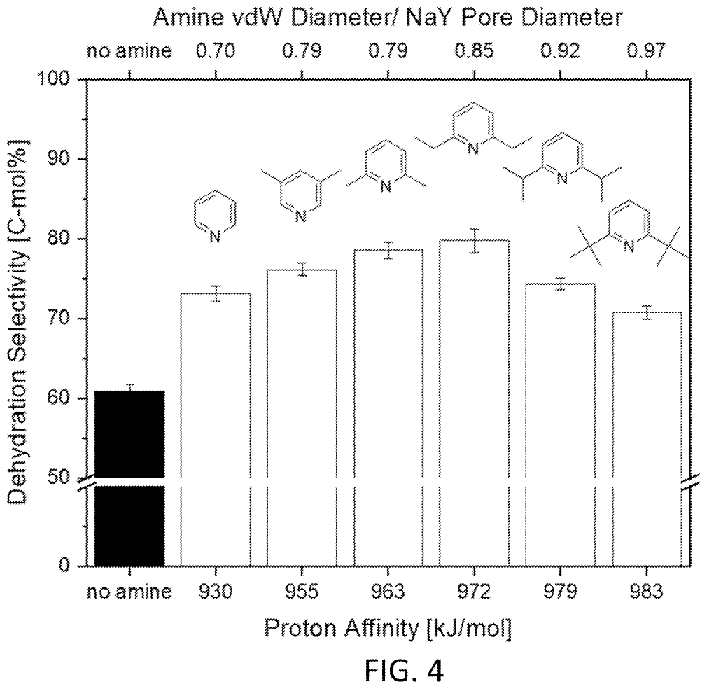

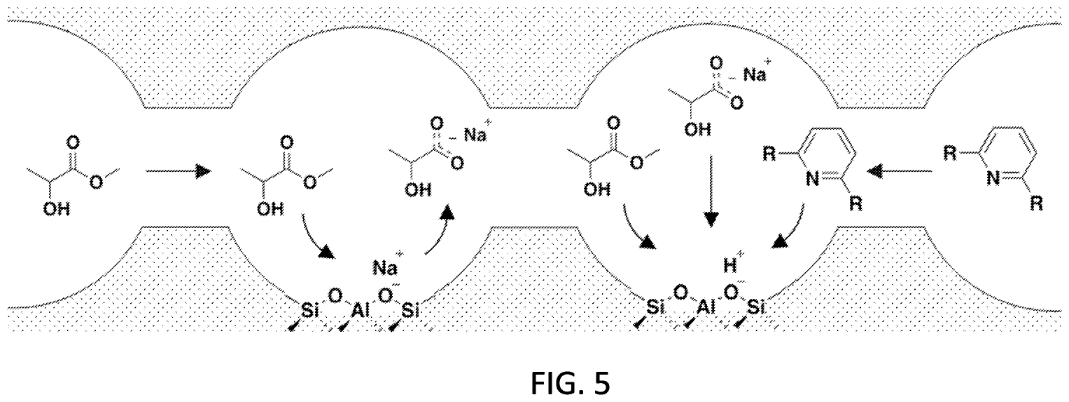

### DETAILED DESCRIPTION

Synthesizing a product including acrylic acid, an acrylic acid ester, an acrylic acid salt, or a combination thereof is achieved by dehydrating a reactant including lactic acid, a lactic acid ester, a lactic acid salt, or a combination thereof. Dehydrating the reactant includes contacting a solid acid catalyst with the reactant. The solid acid catalyst includes surfaces defining pores and a multiplicity of acid sites on the surfaces. The acid sites can be Bronsted acid sites or ion-exchanged acid sites. A multifunctional component is coupled to the surfaces of the solid acid catalyst. Each multifunctional component includes at least two functional groups, and each functional group is configured to accept a proton from an acid site of the multiplicity of acid sites. Such functional groups include but are not limited to amine, imine, alcohol, ether, halide, alkyl halide, thiol, and sulfide groups. The reactant is dehydrated to yield a product including acrylic acid, an acrylic acid ester, an acrylic acid salt, or a combination thereof.

Coupling of the multifunctional component and the solid acid catalyst may occur via absorption of the multifunctional component onto the solid acid catalyst, via solvent mediated impregnation of the multifunctional component onto the solid acid catalyst, via mixing of the multifunctional component with the solid acid catalyst, or via other methods. In some cases, the multifunctional component is provided to a reaction mixture that includes the solid acid catalyst and the reactant. In some cases, the solid acid catalyst is pre-loaded with the multifunctional component to yield a modified catalyst including the solid acid catalyst and the multifunctional component adsorbed or coupled to the surfaces of the solid acid catalyst. As used herein, “coupled” generally refers to a force (e.g., van der Waals) or bond (e.g., ionic bond, covalent bond, hydrogen bond) that binds together two atoms or groups of atoms. In some cases, the multifunctional component may comprise a portion of the feed to the catalyst. The coupling of the multifunctional component to the surfaces of the solid acid catalyst may occur in-situ during reaction. When the acid catalyst is pre-loaded with the multifunctional component, the synthesis may proceed with or without addition of the multifunctional component to the reaction mixture.

The solid acid catalyst can comprise a zeolite. Zeolites are crystalline aluminosilicate compositions which are microporous and which are formed from corner sharing AlO₂ and/or SiO₂ tetrahedra. Synthetic zeolites can be prepared via hydrothermal synthesis employing suitable sources of Si, Al, and structure-directing agents such as alkali metals, alkaline earth metals, amines, and/or organoammonium cations. The structure-directing agents reside in the pores of the zeolite and influence the particular structure of the resulting zeolite. These species balance the framework charge associated with aluminum and can also serve as space fillers. Zeolites are characterized by having pore openings of uniform dimensions, having a significant ion exchange capacity, and being capable of reversibly desorbing an adsorbed phase which is dispersed throughout the internal voids of the crystal without significantly displacing any atoms which make up the permanent zeolite crystal structure.

As used herein, zeolites may be referred to by proper name, such as Zeolite Y (described in J. Chem. Soc., Faraday Trans. I, 1976, 72, 1877-1883) or LZ-210 (described in U.S. Pat. No. 4,503,023), or by structure type code, such as FAU. These three-letter codes indicate atomic connectivity and hence pore size, shape, and connectivity for the various known zeolites. The list of these codes may be found in the Atlas of Zeolite Framework Types, which is maintained by the International Zeolite Association Structure Commission at iza-structure.org/databases/.

Channel systems for known zeolites are described in the Atlas of Zeolite Framework Types as having zero-dimensional, one-dimensional, two-dimensional or three-dimensional pore systems. A zero-dimensional pore system has no pore system running through the zeolite crystal, instead only possessing internal cages. A one-dimensional pore system contains a pore delimited by 8-membered rings or larger that run substantially down a single axis of a crystal. Two-dimensional pore (channel) containing zeolites contain intersecting pores that extend through two-dimensions of a zeolite crystal, but travel from one side of the third dimension of the zeolite crystal to the other side of the third dimension is not possible, while zeolites containing three-dimensional channel systems have a system of pores intersecting, often in a mutually orthogonal manner, such that travel from any side of a zeolite crystal to another is possible. FAU is a three-dimensional zeolite comprising cages and pore openings delimited by 12-membered rings.

In some cases, the zeolite may comprise 12-membered ring pore openings. In some cases, the zeolite comprises one or more of FAU, EMT, MFI, BEA, MOR, LTL, MAZ, SFV, STO, and SAO. In some cases, the zeolite comprises one or more of FAU, EMT, BEA, MOR, LTL, MAZ, SFV, STO, and SAO.

In some cases, the solid acid catalyst includes one or more of silica-alumina, MCM-41, silica gel, metal organic frameworks, and covalent organic frameworks. In one example, the solid acid catalyst is a metal organic framework, and the metal organic framework is a zeolitic imidazolate framework. The solid acid catalyst typically includes aluminum (e.g., at least 0.00001, 0.0001, 0.001, 0.1, or 1 wt % aluminum). In some cases, the solid acid catalyst includes one or more of Na, Li, K, Rb, Cs, Cu, Fe, Co, La, Ce, Sm, Eu, Ca, Sr, and Ba.

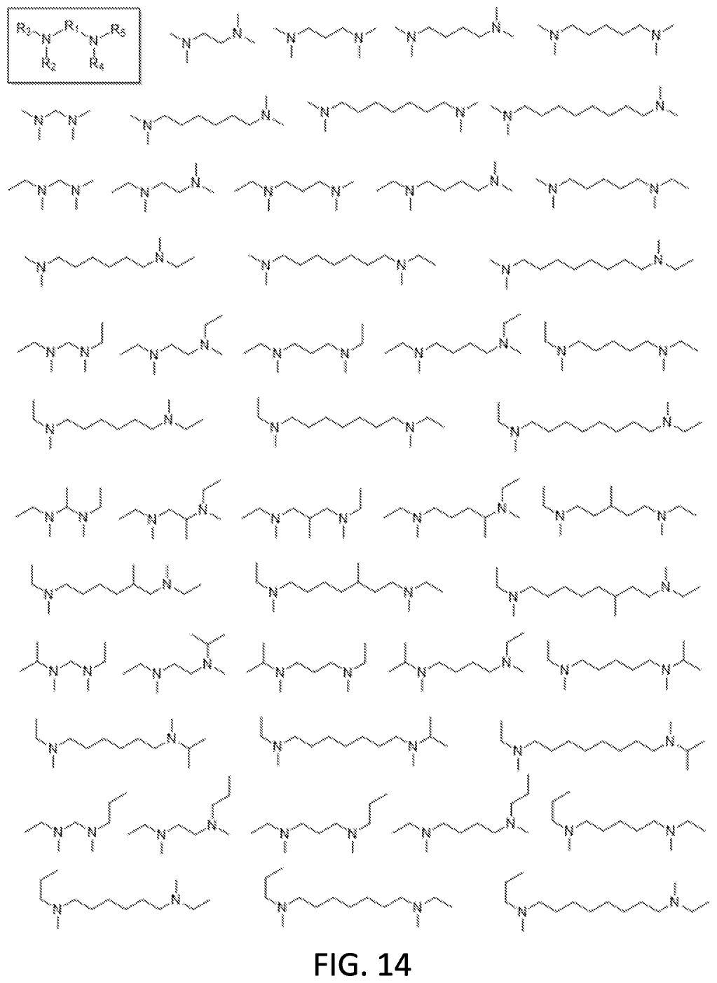

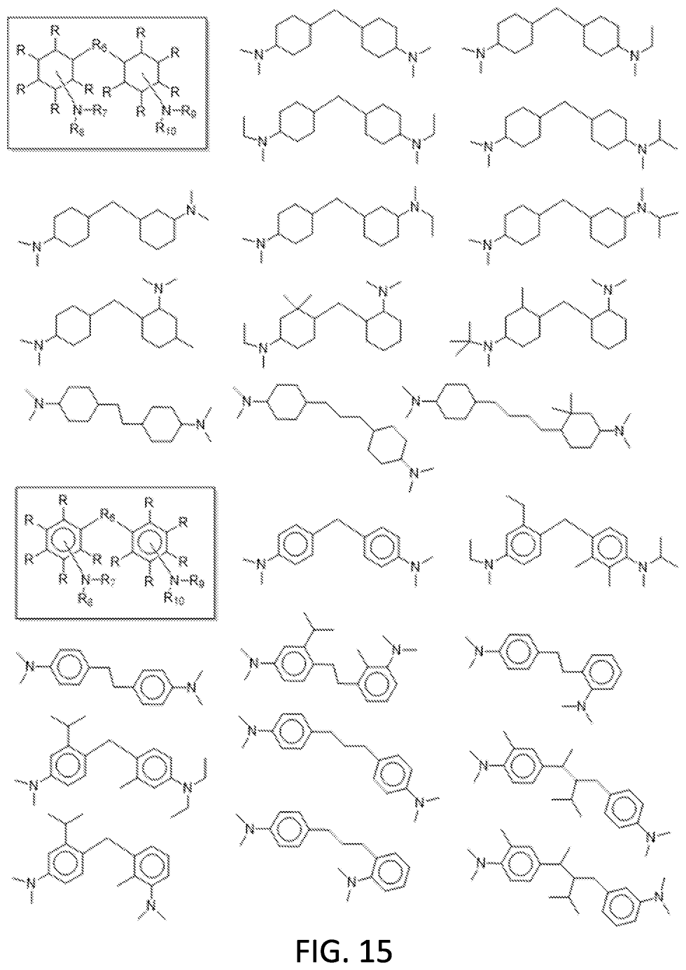

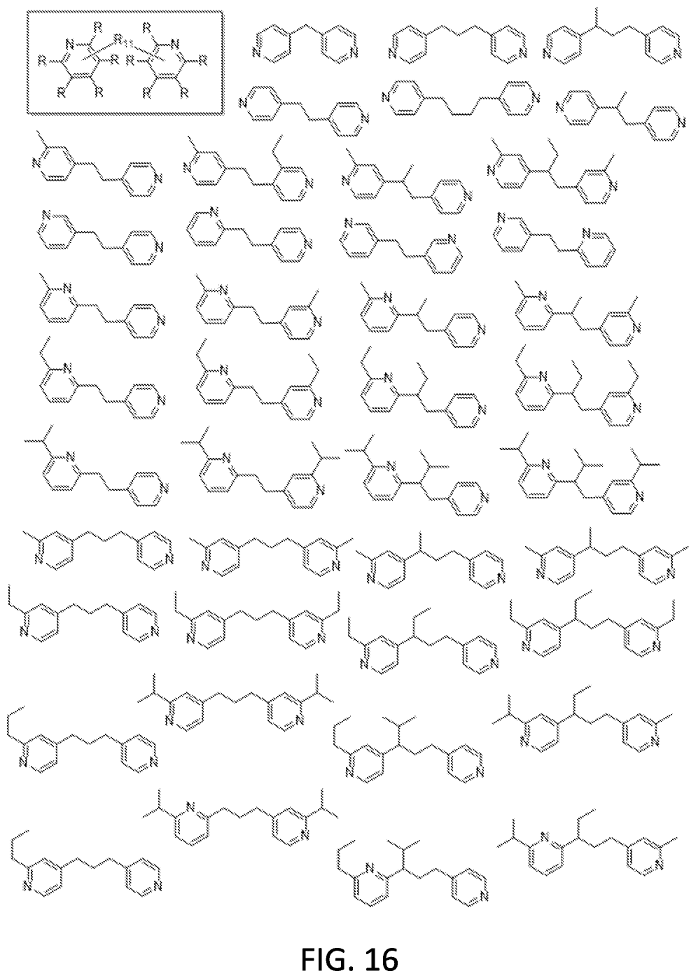

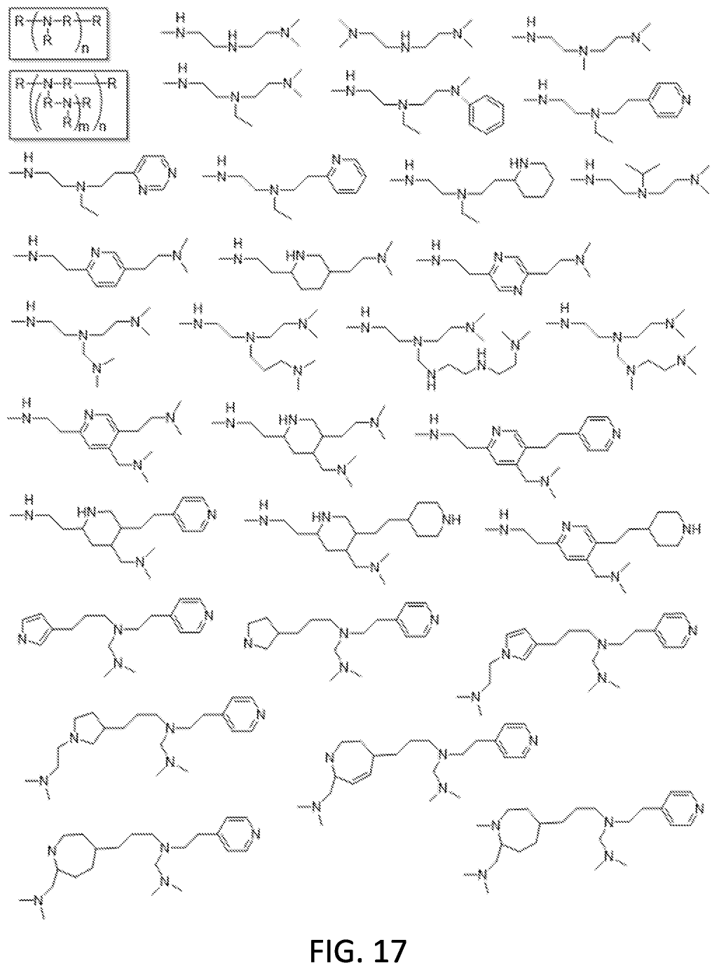

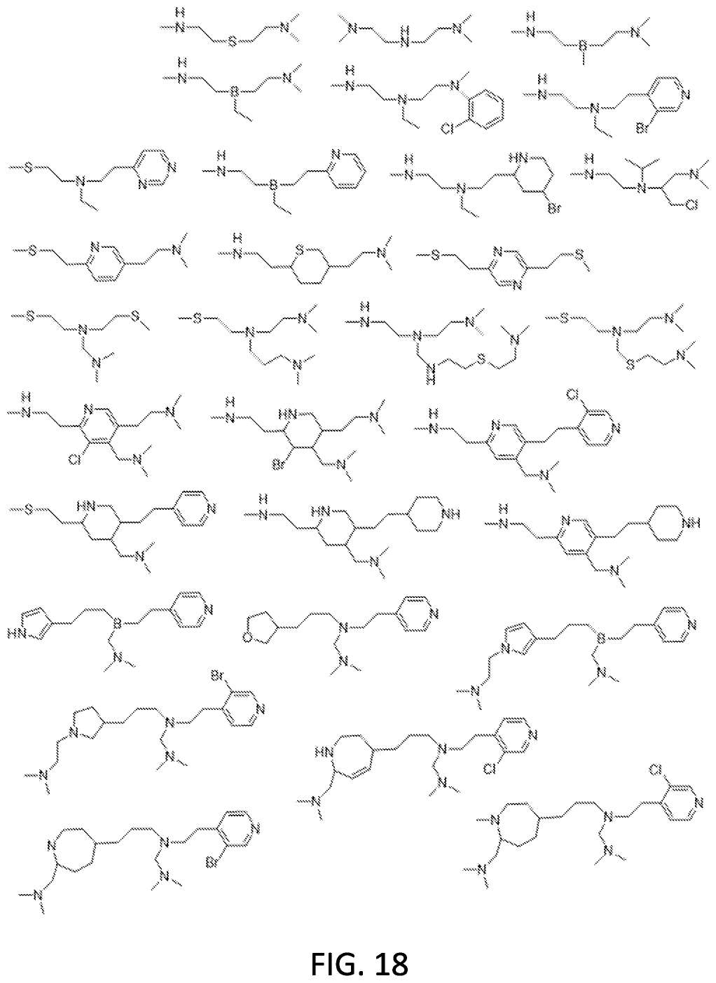

At least one of the at least two functional groups of the multifunctional component is an amine functional group. In some cases, at least two of the at least two functional groups are amine functional groups. Examples of suitable multifunctional components are shown in FIGS. 14-18. In FIG. 14, Class I compounds are depicted wherein each of R₁-R₅ independently represents an alkyl, heteroalkyl, alkene, or heteroalkene group with at least 1 and less than 20 carbon atoms, and each heteroalkyl or heteroalkene group independently includes one or more of S, Cl, Br, B, F, Si, P, N, and O. In an aspect, Class I compounds may comprise at least 4 carbon atoms or may comprise fewer than 15 carbon atoms. Class I compounds may comprise diamines. In FIG. 15, Class II and Class III compounds are depicted wherein each of R₆-R₁₀ independently represents an alkyl, heteroalkyl, alkene, or heteroalkene group with at least 1 and less than 20 carbon atoms, and each heteroalkyl or heteroalkene group independently includes one or more of S, Cl, Br, B, F, Si, P, N, and O. In an aspect, Class II and/or Class III compounds may comprise at least 4 carbon atoms or may comprise fewer than 15 carbon atoms. Class II and/or Class III compounds may comprise diamines. Class II compounds may comprise a cycloalkyl group. Class III compounds may comprise an aromatic group. In FIG. 16, Class IV compounds are depicted wherein each R independently represents H, methyl, ethyl, propyl, or isopropyl, and R₁₁ independently represents an alkyl, heteroalkyl, alkene, or heteroalkene group with at least 1 and less than 20 carbon atoms, and each heteroalkyl or heteroalkene group independently includes one or more of S, Cl, Br, B, F, Si, P, N, and O. In an aspect, Class IV compounds may comprise at least 10 carbon atoms or may comprise fewer than 18 carbon atoms. Class IV compounds may comprise dipyridines. In FIG. 17, Class V and Class VI compounds are depicted wherein each R independently represents an alkyl, heteroalkyl, alkene, or heteroalkene group with at least 1 and less than 20 carbon atoms, and each heteroalkyl or heteroalkene group independently includes one or more of S, Cl, Br, B, F, Si, P, N, and O. Class V and/or Class VI compounds may comprise at least 4 carbon atoms or may comprise fewer than 15 carbon atoms. Class V and/or Class VI compounds may comprise at least 2 nitrogen atoms or may comprise fewer than 5 nitrogen atoms. Class V and/or Class VI compounds may comprise polyamines. Additional examples of suitable multifunctional components include the compounds depicted in FIG. 18.

The at least two functional groups are separated by a spacer. Suitable spacers include an alkyl group, a heteroalkyl group, an alkenyl group, a heteroalkenyl group, a cycloalkyl group, a heterocyclyl group, an aryl group, or a heteroaryl group. The alkyl group, the heteroalkyl group, the alkenyl group, or the heteroalkenyl group typically includes 1-20 carbon atoms (e.g., at least one carbon atom or at least two carbon atoms). The heteroalkyl group or the heteroalkenyl group typically includes one or more of S, Cl, Br, B, F, Si, P, N, and O. The selectivity of the dehydrating reaction may be at least 80 C-mol %, at least 85 C-mol %, or at least 90 C-mol %.

Synthesizing the product can include providing a fluid including the multifunctional component to a vessel containing the solid acid catalyst. The fluid can include a gas or a liquid. When the fluid includes a liquid, synthesizing the product can include vaporizing the liquid. Contacting the solid acid catalyst with the reactant comprises providing a fluid comprising a solvent and the reactant to a vessel containing the solid acid catalyst. Contacting the solid acid catalyst or the modified solid acid catalyst with the reactant may comprise providing a fluid comprising a solvent and reactant to a vessel containing the solid acid catalyst. The solvent may comprise water, an alcohol, an ester, or combinations thereof. A concentration of the reactant may be at least 0.0001, 1, 4, or 10 wt % and less than 50, 75, 90, or 100 wt %. A partial pressure of the reactant is typically in a range between about 0.01, 1, 10, or 100 Pa and about 500, 1000, 2500, or 5000 Pa. Dehydrating the reactant occurs at a reaction temperature between about 50, 100, 150, 200, or 250° C. and about 350, 400, or 500° C. Dehydrating the reactant occurs in a vessel, and a weighted hourly space velocity (WHSV) as defined in Equation 1, of the reactant is in a range between 0.001, 0.1, or 1 and 10, 50, or 100 h⁻¹.

$$WHSV = \frac{mass flowrate of reactant}{mass of catalyst} \qquad ( 1 )$$

A molar ratio of the multifunctional component to the reactant, namely the lactate or lactic acid, is typically in a range between about 0.001, 0.01, 0.1, or 1 to about 10, 20, 50, 100, 250, 500, or 1000. A weight ratio of the multifunctional component to the solid acid catalyst during reaction is in a range between about 0.001, 0.1, 0.25, or 0.5 wt % and about 5, 10, 50, or 100 wt %.

During synthesis of the product, a reaction mixture typically includes the solid acid catalyst defining pores and having acid sites (e.g., Bronsted acid sites, ion-exchanged acid sites, or both) on surfaces defining the pores, the multifunctional component having at least two functional groups, and the reactant including lactic acid, a lactic acid ester, a lactic acid salt, or a combination thereof. Each functional group is configured to accept a proton from one of the acid sites. The reaction mixture can also include acrylic acid, acrylic acid ester, acrylic acid salt, or a combination thereof. A temperature of the reaction mixture is typically in a range between about 50° C. and about 500° C.

### EXAMPLES

Lactic Acid and Lactate Ester Dehydration Reaction Activity Measurements.

All reactivity measurements were performed at atmospheric pressure using a micro-flow catalytic packed bed reactor contained in a modified gas chromatography (GC) inlet. The temperature and gas flowrate were precisely controlled by the Agilent 7890B GC. The catalyst bed was comprised of 20 mg of ex-situ calcined zeolite sample sandwiched between two layers of deactivated quartz wool (Restek, CAS. #20789). Prior to reaction, the catalyst bed was calcined in situ with air at 400° C. for five hours with a ramp rate of 3 C min⁻¹. A continuous liquid reactant flow was fed by a Cole-Parmer 78-8110C syringe pump to a vaporizer embedded in the valve box of the GC, where the liquid reactant feed was vaporized and carried by the carrier gas helium to other gas lines in the reactor. Similarly, titrant amines were introduced to the reactor system using a separate Cole-Parmer 78-8110C syringe pump. The feed streams containing reactant and titrant were directed using the switching valve in GC to flow through or bypass the catalyst bed.

The reactor effluent was separated by an HP-FFAP column (Agilent Technologies) with a ramp rate of 10° C. min⁻¹ from 50° C. to 240° C. and analyzed by a quantitative carbon detector (QCD, Polyarc™) in conjunction with a flame ionization detector (FID). Due to the QCD, the molar flowrate of carbons associated with each species can be directly measured using one pre-determined calibration factor. Prior to material characterization and reactivity measurements, NaY (Zeolyst International, CBV-100, Si/Al=2.6, lot #100031080671) and NH4⁺-Y (Zeolyst International, CBV-500, Si/Al=2.6, lot #400054002618) zeolite catalysts were calcined at 550° C. for 12 h with a 1.0° C./min ramp rate to remove water and residual organic compounds. CBV-500 was originally in the ammonium form and was converted into proton form (HY) after calcination. All chemicals, namely, HPLC-grade water, methyl lactate [98%], acetaldehyde [99.5%], methanol [99.9%], methyl acrylate [99%], 2,3-pentanedione [97%], methyl pyruvate [95%], acrylic acid [99%], pyridine [99.8%], 3,5-dimethylpyridine [98%], 2,6-dimethylpyridine [99%], 2,6-diethylpyridine [95%], 2,6-diiso-propylpyridine [97%], 2,6-ditertbutylpyridine [97%], 1,2-dimethylimidazole [98%], N,N,N′,N′-tetramethylethylenediamine [99.5%], and 1,1,3,3-tetramethylguanidine [99%] were purchased from Sigma-Aldrich and used as received.

Amine Co-Feed Experiments.

Methyl lactate dehydration was evaluated over Na-FAU with co-fed amines at 300° C. Na-FAU was loaded to the catalyst bed and 30 wt % methyl lactate aqueous solution was fed to the reactor at a flowrate of 1.0 μL min⁻¹. Amine was introduced in a separate feed stream to the reactor with a flowrate to attain a 10:1 ratio of methyl lactate:amine functional group. At the beginning of each experiment, four bypass runs were performed and averaged to measure the total molar flowrate of carbons from methyl lactate, Ń_{MLO}, entering the reactor system. The carbon balance was defined by Equation (2),

$$carbon balance = \frac{{\dot{N}}_{CO} + {\dot{N}}_{A C} + {\dot{N}}_{MT} + {\dot{N}}_{MA} + {\dot{N}}_{PD} + {\dot{N}}_{MP} + {\dot{N}}_{ML} + {\dot{N}}_{AA}}{{\dot{N}}_{MLo}} \times 100 \% \qquad ( 2 )$$

where AC is acetaldehyde, MT is methanol, MA is methyl acrylate, PD and MP are minor side products, 2,3-pentanedione and methyl pyruvate, respectively, ML is methyl lactate, and AA is acrylic acid. All the molar flowrates are carbon molar flowrates of the corresponding species. The carbon-based conversion and molar reaction rate were calculated as:

$$conversion = \frac{{\dot{N}}_{CO} + {\dot{N}}_{A C} + {\dot{N}}_{MT} + {\dot{N}}_{MA} + {\dot{N}}_{PD} + {\dot{N}}_{MP} + {\dot{N}}_{AA}}{{\dot{N}}_{MLo}} \times 100 \% \qquad ( 3 )$$

$${rate}_{i} = \frac{\frac{{N}_{i}}{number of carbons in i}}{mass of caalyst} , i represents species i in the system . \qquad ( 4 )$$

Carbon balances, unless otherwise stated, for all experiments are 100±10 wt % during the run. Productivity was reported instead of rate using the same Equation (4) when the reactions were not operated under differential conditions. The dehydration pathway selectivity (or dehydration selectivity) was calculated as:

$$dehydration pathway selectivity = \frac{{\dot{N}}_{MA} + {\dot{N}}_{AA} + {\dot{N}}_{MT \_DH}}{{\dot{N}}_{CO} + {\dot{N}}_{A C} + {\dot{N}}_{MT} + {\dot{N}}_{MA} + {\dot{N}}_{PD} + {\dot{N}}_{MP} + {\dot{N}}_{AA}} \times 100 \% \qquad ( 5 )$$

\dot{N}_{MT_DH} is the molar flowrate of carbons of methanol from the dehydration pathway and is assumed to be

$$\frac{1}{3} {\dot{N}}_{AA} .$$

The carbon balance on methanol shows that methanol was conserved in the form of methyl acrylate, methyl lactate, methyl pyruvate, and methanol itself.

The decrease in conversion over time or catalyst deactivation was evaluated with a first-order deactivation model as shown in Equation (6).

ln ln(1/1−X(t))=−kt+δ (6)

X(t) is the methyl lactate conversion at time t and X₀ is the initial conversion. k is the deactivation time constant that characterizes the rate of deactivation.

Amine Pre-Saturated Experiments.

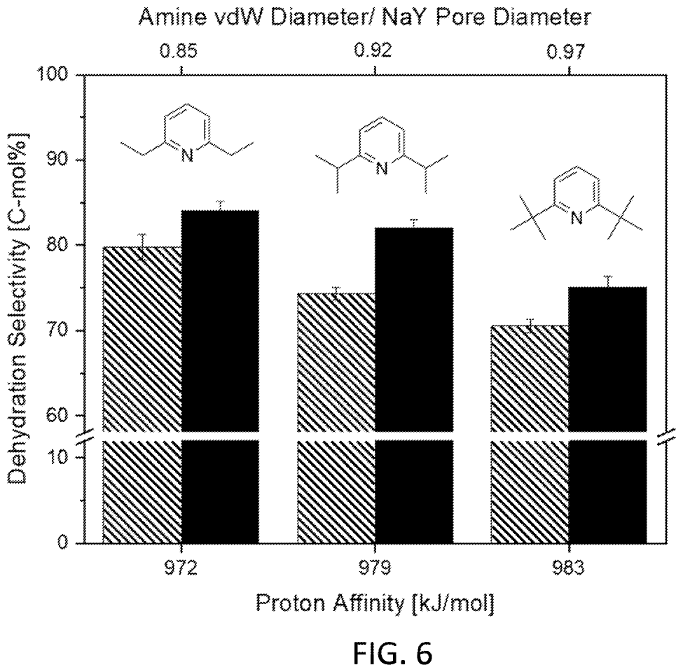

For the experiments presented in FIG. 6, the Na-FAU catalyst bed was pre-saturated with amine by flowing amine through the catalyst bed for 24 hours with the same flowrate as in the co-feed experiments. After pre-saturation, methyl lactate was co-fed with amine over Na-FAU at the molar ratio of 10:1 of methyl lactate: amine at the same flowrates as in the co-feed experiments. The resulting reactivities were measured and calculated as described herein.

In-Situ Titration Experiments.

Bronsted acid site density of H-FAU and Na-FAU was also determined by in-situ titration of methanol dehydration using 2,6-dimethylpyridine as the titrant. Pyridine has been commonly used as the in-situ titrant for Bronsted acid site measurements in porous materials. However, pyridine is not Bronsted acid site specific and overestimates Bronsted acid density due to nonselective adsorption on both Bronsted acid sites and Lewis acid sites. 2,6-dimethylpyridine shows selective Bronsted acid site adsorption due to the methyl substituent groups at the two and six positions of the pyridine ring. Therefore, 2,6-dimethylpyridine was selected as the titrant for Bronsted acid site estimations. The in-situ titration measurements were performed on the reactor setup described herein. While 2,6-dimethylpyridine desorbs at 240° C. from sodium acidic sites on Na-FAU, the desorption temperature from Bronsted acidic sites on H-FAU is expected to be higher than 240° C. due to formation of hydrogen bonding. The desorption temperature is also expected to be higher than 240 C for amines with higher basicity than 2,6-dimethylpyridine. Therefore, a reaction temperature below 240° C. promotes irreversible adsorption on Bronsted acid sites for amines with the same or higher basicity than 2,6-dimethylpyridine.

For the H-FAU catalyst sample, methanol was delivered at 150° C. at a partial pressure of 0.3 kPa to achieve differential conversion at ˜6%. Dimethyl ether was the only observed product from methanol on H-FAU at the specified reaction temperature. At steady conversion and dimethyl ether synthesis rate, 7.8 Pa of 2,6-dimethylpyridine was introduced to quench methanol dehydration. The cumulative amine uptake was calculated from the amine flowrate exiting the H-FAU catalyst bed. When amine adsorption reached saturation, the rate of dimethyl ether formation remained constant, and the cumulative amine uptake at saturation yielded an estimate for the Bronsted acid site density of H-FAU. Methanol was also fed to 20 mg of Na-FAU at a partial pressure of 0.3 kPa. The conversion of methanol on Na-FAU was explored at 150, 180, 230, and 300° C. prior to the introduction of 2,6-dimethylpyridine to measure Bronsted acid site count.

The Bronsted acid site accessibility by amine under competition with methyl lactate was measured with in-situ titration experiments using methyl lactate decarbonylation as the probe reaction. The proton form of zeolite Y (H-FAU) with the same Si/Al ratio as that of Na-FAU was used to directly estimate the competition between methyl lactate displacement and amine adsorption on Bronsted acid sites. The reaction temperature was 180° C. to achieve irreversible amine adsorption and measurable methyl lactate decarbonylation activities. The partial pressure of methyl lactate and water as well as the weight hourly space velocity (WHSV) remained the same as in the amine co-feed experiments. Methyl lactate flowed through the H-FAU catalyst bed at 180° until conversion fluctuated only by ˜1% within one hour. A steady stream of amine was then introduced at a molar ratio of methyl lactate:amine of 240:1. The reaction was allowed to proceed until a new steady state conversion was reached with fluctuation within 0.1% for one hour.

Results.

The effect of amine methyl lactate dehydration selectivity improvement was evaluated by co-feeding amines with different basicity and size over Na-FAU catalyst. Amines with high basicity and moderate size or steric limitations afford increased selectivity control.

Materials Characterization.

The pore diameter and volume from argon physisorption for Na-FAU and H-FAU are listed in Table 1. Solid-state ²⁷Al-MAS NMR indicated negligible presence of extra-framework aluminum in Na-FAU. ICP results revealed the actual Si/Al ratio to be 2.65 for Na-FAU and 2.70 for H-FAU. Reactive gas chromatography (RGC) was used to measure Bronsted acid site densities of the solid acid catalysts. Reactions were carried out in a microcatalytic reactor housed within a temperature-controlled GC inlet liner, and products were then separated in a GC column and quantified by a flame ionization detector. RGC indicated negligible Bronsted acidity in Na-FAU, which was supported by the absence of methanol conversion on Na-FAU for a wide range of temperatures from 150 to 300° C. The Bronsted acid site density of H-FAU was determined to be 1250 μmol g⁻¹ by in-situ titration on methanol dehydration.

TABLE 1
The Si/Al ratio and textural information of zeolite samples
measured by ICP and argon physisorption at 87 K.

| | Na-FAU | H-FAU |
|---|---|---|
| Co-fed Amine | (CBV-100) | (CBV-500) |
| Si/Al | 2.65 | 2.70 |
| Pore Diameter [Å] | 7.17 | 7.45 |
| Pore Volume | 0.440 | 0.286 |
| [cm³ g⁻¹] | | |

Selectivity Control of Amines and Na-FAU Catalyst Stability.

Amines with higher basicity than pyridine were selected, as characterized by the tabulated proton affinity values. Here, basicity is varied between different amines by adding electron-donating alkyl substituent groups on pyridine. Alkyl groups with more carbons are more electron donating and result in a more basic amine. For example, the proton affinity of 2,6-dimethylpyridine (2,6DMP) is 33 kJ/mol higher than that of pyridine (Py) and 9 kJ/mol lower than that of 2,6-diethylpyridine (2,6DEP) as shown in FIG. 2. Addition of more carbons to the alkyl groups results in a further increase in proton affinity for 2,6-diisopropylpyridine (2,6DIPP) and 2,6-ditertbutylpyridine (2,6DTBP). Furthermore, amines with alkyl groups closer to the pyridine nitrogen exhibit higher basicity as shown by the proton affinity increase with methyl groups at the three and five positions on 3,5-dimethylpyridine (3,5-DMP) to the two and six positions on 2,6-DMP.

Without being bound by theory, a structure-property relationship may be expected between the amine and solid acid. The size descriptor selected for the titrant amines was the van der Waals (vdW) diameter. 2,6-DIPP and 2,6-DTBP are estimated by the van der Waals diameter to barely fit in the Na-FAU pores and may experience steric hindrance in FAU. This is consistent with results described herein that address the steric effects associated with these two bulky amines. Therefore, amine size was characterized using van der Waals diameter.

TABLE 2
Size of methyl lactate and selected amines was estimated using effective
diameter, maximum diameter, van der Waals diameter, and kinetic diameter.

| | | Effective | Maximum | van der Waals | Kinetic |
|---|---|---|---|---|---|
| | | Diameter | Diameter | Diameter | Diameter |
| Amine | CAS # | (Å) | (Å) | (Å) | (Å) |
| pyridine | 110-86-1 | 6.36 | 7.10 | 5.00 | 5.30 |
| methyl lactate | 27871-49-4 | 6.09 | 8.06 | 5.34 | 5.18 |
| 3,5-dimethylpyridine | 591-22-0 | 6.46 | 8.74 | 5.63 | 5.86 |
| 2,6-dimethylpyridine | 108-48-5 | 7.20 | 8.66 | 5.63 | 5.86 |
| triethylamine | 121-44-8 | 7.28 | 8.29 | 5.83 | 5.75 |
| 2,6-diethylpyridine | 935-28-4 | 7.36 | 11.4 | 6.12 | 6.33 |
| 4-tert-butylpyridine | 3978-81-2 | 6.50 | 9.00 | 6.15 | 6.33 |
| tripropylamine | 102-69-2 | 8.46 | 10.3 | 6.53 | 6.46 |
| 2,6-diisopropylpyridine | 6832-21-9 | 7.84 | 10.7 | 6.59 | 6.74 |
| 2,6-di-tert-butylpyridine | 585-48-8 | 8.10 | 11.2 | 6.98 | 7.11 |
| tributylamine | 102-82-9 | 9.64 | 12.2 | 7.11 | 7.04 |

The addition of alkyl groups on pyridine to increase amine basicity inevitably increases the size of amines FAU topology comprises a large central cavity called the ‘supercage’, which has a diameter of 11.2 Å. Connections of supercages form channels or pores in FAU. The pores are delimited by 12-membered rings which have a diameter of about 7.4 Å, which agrees with the 7.17 Å Na-FAU pore diameter measured by argon physisorption. A non-dimensional amine size was then calculated by normalizing the calculated amine van der Waals diameters by the experimentally measured Na-FAU pore diameter. As the non-dimensional molecular size approaches 1.0, steric hindrance occurs due to the bulkiness of the amine and the rigidity of both the zeolite framework and the pyridine ring. Larger molecules will be sieved, preventing access to active sites.

In a comparative example, the effect of the amine on methyl lactate dehydration was first evaluated by co-feeding pyridine (Py), 3,5DMP, 2,6DMP, and 2,6DEP at a molar ratio of methyl lactate:amine of 10:1 over Na-FAU. At 300° C., methyl lactate dehydration selectivity was observed to increase by 12.3-19.1 C-mol % with the addition of these amines at initial conversion in the range of 55.4 to 91.5 C-mol % (Table 3). Higher dehydration selectivities were measured for amines with higher basicities. The productivity of acetaldehyde, a byproduct from the decarbonylation pathway, was suppressed to almost zero with the introduction of amines, indicating that the improvement in dehydration selectivity may be due to the suppression on the major side reaction, methyl lactate decarbonylation. Without being bound by theory, the ability to titrate in-situ Bronsted acid sites and suppress decarbonylation may be a factor in controlling the selectivity of methyl lactate dehydration.

TABLE 3
Reactivity of methyl lactate over Na-FAU with the addition
of amines is reported at reaction condition of 300° C.,
Pₜₒₜₐₗ = 101.3 kPa (P_{methyl lactate} = 78.0 Pa, Pₐₘᵢₙₑ = 7.8 Pa,
P_{water} = 1.1 kPa, balanced by He), and WHSV = 0.9
h⁻¹. Error bars are 95% confidence intervals estimated
from error propagation.

| | Proton | Dehydration | Initial |
|---|---|---|---|
| | Affinity | Selectivity | Conversion |
| Co-fed Amine | [kJ/mol] | [C-mol %] | [C-mol %] |
| No Amine | — | 60.9 ± 1.0 | 91.5 ± 2.7 |
| Py (pyridine) | 930 | 73.2 ± 1.0 | 55.4 ± 1.5 |
| 3,5DMP | 955 | 76.1 ± 1.0 | 58.3 ± 1.3 |
| (3,5-dimethylpyridine) | | | |
| 2,6DMP | 963 | 78.5 ± 1.0 | 64.0 ± 1.8 |
| (2,6-dimethylpyridine) | | | |
| 2,6DEP | 972 | 80.0 ± 1.5 | 64.0 ± 2.5 |
| (2,6-diethylpyridine) | | | |

In addition to increasing dehydration selectivity, improvement in catalyst stability may also be observed with the introduction of amines. Methyl lactate conversion decreased over time with or without the addition of amines at 300° C., which indicated catalyst deactivation. The deactivation time constant (k) characterizing the rate of deactivation was reduced two-fold with added amines, potentially due to decarbonylation suppression.

Without being bound by theory, while external Bronsted acid sites on the outer surface of zeolites are in general accessible to both reactants and titrants regardless of their overall size, accessing Bronsted acid sites in zeolite pores requires molecules to transport through zeolite pores via internal diffusion. Therefore, the first barrier for amine titration is the internal diffusion limit, which is governed by the overall steric limitations of the amine. Amines with a larger overall size and a higher degree of steric hindrance must pass through the windows between supercages and diffuse slower in zeolite pores, which reduces the effectiveness of Bronsted acid site titration by amines. Note that methyl lactate can readily access Bronsted acid sites faster than bulky amines. Under co-feed conditions, methyl lactate displacement can occur before bulky amines can access Bronsted acid sites due to internal diffusion limitations, resulting in ineffective Bronsted acid site titration. However, internal diffusion limitations may be overcome by allowing enough time for the bulky amine to diffuse through Na-FAU pores.

Once the amine approaches a Bronsted acid site, it will then undergo competitive adsorption with methyl lactate on the Bronsted acid site. As amines approach Bronsted acid sites, proton transfer between the site and amine would yield an ion pair between the protonated amine and the deprotonated zeolite, which stabilizes the protonated amine via electrostatic interactions. When steric repulsion from large substituent groups such as the tert-butyl groups in 2,6DTBP is significant, adsorption is expected to be less favorable.

Upon adsorption on a Bronsted acid site, the tendency for amine desorption depends on the binding strength of the particular amine. The binding strength is governed by both the basicity and local sterics of the adsorbate molecule. Open sites may then be captured by methyl lactate, which may lead to side reactions and reduces dehydration selectivity.

As the overall size of amine increases with respect to the pore size of the porous catalysts, internal diffusion limitations may hinder the effectiveness of amine titration.

The potential effect of preparation method was probed by experiment on the reduction of hindrance from internal diffusion limitations. Na-FAU was pre-saturated with flowing amine through the catalyst bed for 24 hours, which ensured sufficient time for even 2,6DTBP to completely diffuse through Na-FAU pores. After the pre-saturating treatment, methyl lactate and the amine were co-fed to Na-FAU at 300° C. with the same WHSV and molar ratio as used in the previous co-feed experiment. Pre-saturating the catalyst bed with bulky amines (i.e., 2,6DEP, 2,6DIPP, and 2,6DTBP) was observed to significantly improve methyl lactate dehydration selectivity, as shown in FIG. 6. The increase in dehydration selectivity may indicate enhancement in Bronsted acid site titration. By properly selecting treatment conditions and amine, the effect of internal diffusion limitations can be significantly reduced on dehydration selectivity control. A dehydration selectivity as high as 84.1% was experimentally measured with 2,6DEP under pre-saturated condition (black) as a comparative example (diagonal) (FIG. 6). The dehydration selectivity with 2,6DIPP under pre-saturated conditions is comparable to the selectivity for 2,6DEP under co-feed or pre-saturated conditions. However, the improvement in dehydration selectivity with 2,6DTBP is not as apparent as with 2,6DIPP, which indicates the existence of an additional factor that affects Bronsted acid site titration by 2,6DTBP.

Without being bound by theory, it is believed that an amine adsorption process may occur in three steps: (i) zeolite deprotonation, (ii) amine protonation, and (iii) the stabilization interaction between the protonated amine and the deprotonated zeolite. The binding energy can be calculated as the energy difference between the final optimized adsorbed structure and the initial structures of amine and the empty zeolite at their fully relaxed states. The deprotonation energy (DPE) of zeolite Y has been calculated to be 1200 kJ/mol, and the proton affinity (PAFF) values for amines have been experimentally measured. The stabilization energy (E_{stab}) can then be calculated by Equation (7).

E_{stab}=Binding Energy−DPE−PAFF (7)

In addition to escaping from the zeolite wall, bulky amines may also undergo structural change from their fully relaxed structures in order to be accommodated in the constrained zeolite voids. Distortion energy was calculated to determine the energy gain in the structural change of amines. The distortion energy for amines smaller than 2,6DTBP are similar and below 10 kJ/mol, while the distortion energy of 2,6DTBP is 18.6 kJ/mol, indicating a larger structural change is required to form the ion-pair structure. DFT calculations for amine adsorption on FAU provide insight into how local steric limitations impact the binding strength of amine. Local steric constraints destabilize amine binding by reducing the zeolite stabilization and forcing amine to undergo unfavored structural changes to fit in the zeolite void.

In-situ titration experiments revealed Bronsted acid sites accessibility by amines in same environment enriched in methyl lactate and water as in the co-feed experiments. Methyl lactate decarbonylation on H-FAU was titrated by 2,6DMP, 2,6DEP, and 2,6DTBP at 180° C. until saturation. The cumulative amine uptakes are 691, 417, and 136 μmol (g_{cat})⁻¹ for 2,6DMP, 2,6DEP, and 2,6DTBP respectively. The three-fold lower cumulative uptake for 2,6DTBP compared with that of 2,6DEP shows that the Bronsted acid site accessibility is significantly more limited for 2,6DTBP than 2,6DEP. The percentage of productivity suppressed was calculated to be 88%, 81%, and 72% respectively for 2,6DMP, 2,6DEP, and 2,6DTBP by Equation (8).

$$Percentage of productivity suppressed = 1 - \frac{productivity at saturation}{productivity at steady state before titration} \qquad ( 8 )$$

Despite the significant differences in cumulative amine uptake among the three amines, the percentage of productivity suppressed is comparable between 2,6DMP, and 2,6DEP and between 2,6DEP and 2,6DTBP. The suppression of catalyst activities can also be caused by pore blockage in addition to acid site poisoning. As the size of 2,6DTBP is comparable to the window size of zeolite Y, pore blockage by bulky amines can also suppress catalyst activity even with a low amine uptake.

The low calculated binding strength and low site accessibility show that Bronsted acid site adsorption by 2,6DTBP is hindered by the amine local steric limitations. Even given sufficient time to overcome internal diffusion limitations due to overall steric limitations, amines not of the instant invention with high degree of local steric limitations such as 2,6DTBP will still not be able to undergo desirable Brønsted acid site titration.

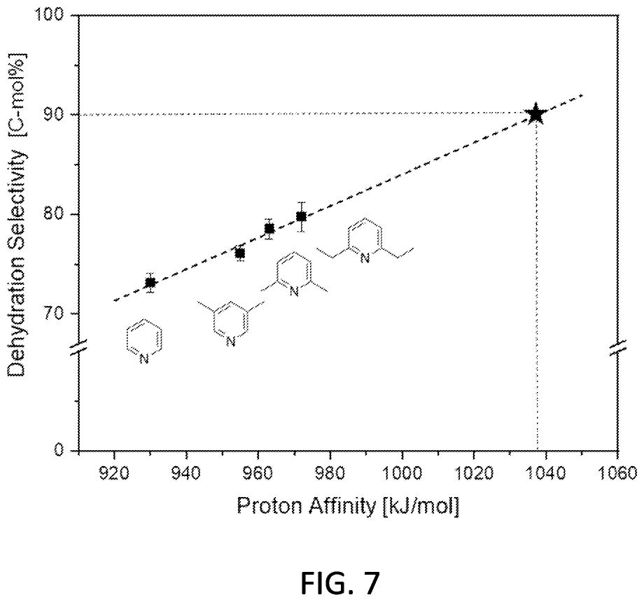

While amine basicity may be utilized for Bronsted acid site titration to eliminate side reactions, amine steric effects remain a limiting factor that may prevent the achievement of greater than 80% selectivity or greater than 85% selectivity or great than 90% selectivity or greater than 92% selectivity in lactate dehydration. Therefore, amines with high basicity but low steric limitations may be desirable for methyl lactate dehydration over porous solid acid catalysts. When the effect of amine steric limitation is minimal, amine basicity is expected to determine Bronsted acid site titration and further improve dehydration selectivity due to the increase in basicity (FIG. 7).

TABLE 4
46 bases with proton affinity higher and van der Waals diameter
smaller than those of 2,6DEP are summarized below.

| | Proton | van der Waals | | Proton | van der Waals |
|---|---|---|---|---|---|
| CAS | Affinity | Diameter | CAS | Affinity | Diameter |
| Number | [kJ/mol] | [Å] | Number | [kJ/mol] | [Å] |
| 113-00-8 | 986.3 | 4.52 | 874-39-5 | 994.6 | 5.87 |
| 1184-78-7 | 983.2 | 5.07 | 80-70-6 | 1031.6 | 5.87 |
| 109-76-2 | 987 | 5.13 | 3001-72-7 | 1038.3 | 5.87 |
| 1606-49-1 | 1002 | 5.16 | 22207-84-7 | 977.2 | 5.91 |
| 504-24-5 | 979.7 | 5.24 | 554-70-1 | 984.5 | 5.94 |
| 6338-45-0 | 976.7 | 5.36 | 45676-04-8 | 987 | 5.94 |
| 10447-93-5 | 977.6 | 5.36 | 14287-89-9 | 978 | 6.00 |
| 13325-10-5 | 984.5 | 5.38 | 45651-41-0 | 979.4 | 6.00 |
| 1739-84-0 | 984.7 | 5.39 | 124-09-4 | 999.5 | 6.00 |
| 110-60-1 | 1005.6 | 5.45 | 71-00-1 | 988 | 6.02 |
| 110-70-3 | 989.2 | 5.46 | 110-18-9 | 1012.8 | 6.02 |
| 2305-59-1 | 988.1 | 5.48 | 695-88-5 | 982.5 | 6.03 |
| 51-45-6 | 999.8 | 5.59 | 5261-65-4 | 986.9 | 6.03 |
| 5397-67-1 | 978 | 5.73 | 4458-31-5 | 978.8 | 6.05 |
| 462-94-2 | 999.6 | 5.74 | 35079-50-6 | 979.9 | 6.05 |
| 109-55-7 | 1025 | 5.75 | 6006-15-1 | 996.4 | 6.05 |
| 100-76-5 | 983.3 | 5.78 | 5807-14-7 | 1054.6 | 6.05 |
| 921-04-0 | 975.9 | 5.82 | 19616-52-5 | 1046.4 | 6.09 |
| 918-02-5 | 979.6 | 5.82 | 935-28-4 | 972.3 | 6.12 |
| 121-44-8 | 981.8 | 5.83 | 6188-30-3 | 996.4 | 6.12 |
| 1122-58-3 | 997.6 | 5.83 | 875-80-9 | 998.2 | 6.12 |
| 933-69-7 | 987.4 | 5.87 | 3268-61-9 | 1000.5 | 6.12 |
| 934-37-2 | 990.9 | 5.87 | 13439-84-4 | 1047.7 | 6.12 |

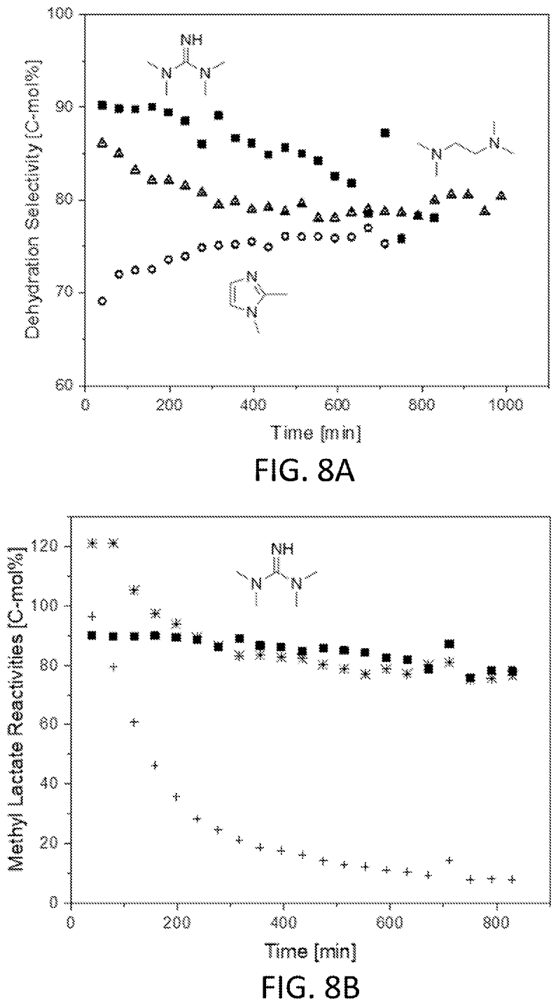

Selected bases were examined for methyl lactate dehydration selectivity enhancement. A dehydration selectivity of 89 C-mol % was observed with 1,1,3,3-tetramethylguanidine (TMG) for the first five hours of co-feed experiment (FIG. 8A). TMG, albeit a strong base, may be unstable under reaction conditions. The approach of using amines with a single basicity group to improve dehydration selectivity requires both high basicity and high stability. However, high basicity often corresponds to high reactivity because the highly basic functional group can act as a strong nucleophile. Therefore, small, nonflexible, but highly basic amines may not be the most desirable solution for methyl lactate dehydration selectivity enhancement.

Dehydration Using Multifunctional Components as Flexible Modifiers.

Multifunctional components with multiple flexible binding sites are shown to result in higher effective binding coverage to acid sites even though the basicity of each binding site does not necessarily exhibit high basicity. The theory can be verified by selecting molecules that have multiple functional groups such as amines tethered together by alkyl chains, among many other possibilities.

Modulation of methyl lactate dehydration used a multifunctional component with multiple basic functional groups, 1,2-bis(4-pyridyl)ethane (1,2BPE), which has two pyridine functional groups connected by an ethyl chain. Reactivity measurements were performed in the same packed bed reactor and under the same reaction conditions as described in previous experiments with single amines. However, unlike monoamines, which usually exist in the liquid phase at room temperature, most diamines, especially dipyridines, are solid under ambient conditions. Therefore, 1,2BPE was dissolved in 30 wt % methyl lactate aqueous solution to achieve a molar ratio of methyl lactate:diamine of 20:1 or a 10:1 ratio of methyl lactate:basic functional group as previously used in comparative experiments with single amines including pyridine.

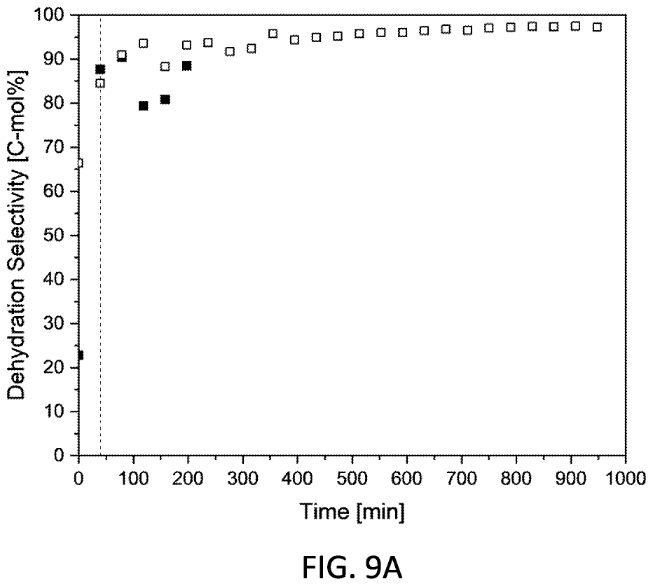

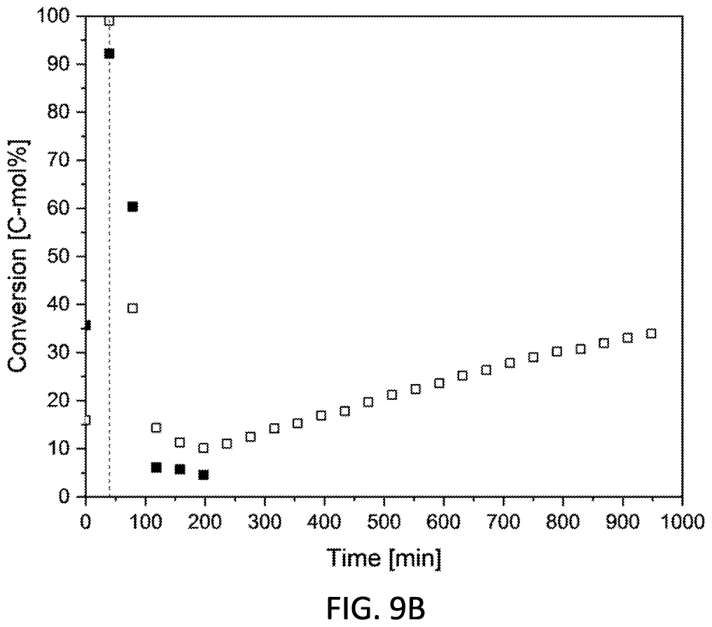

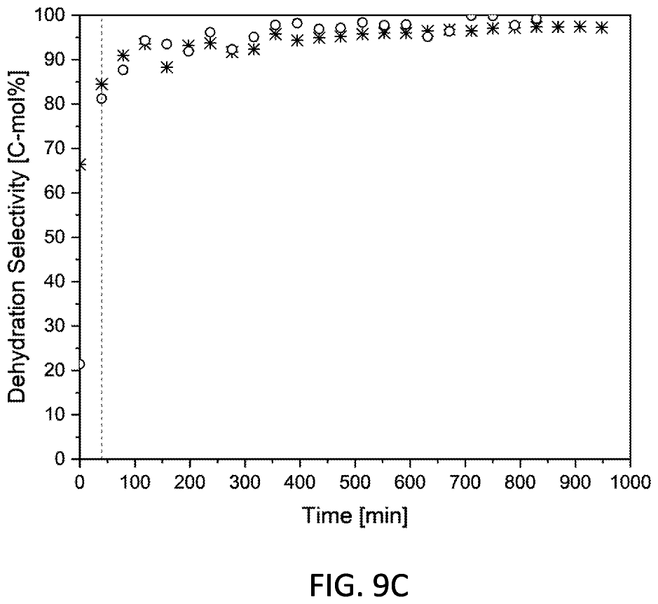

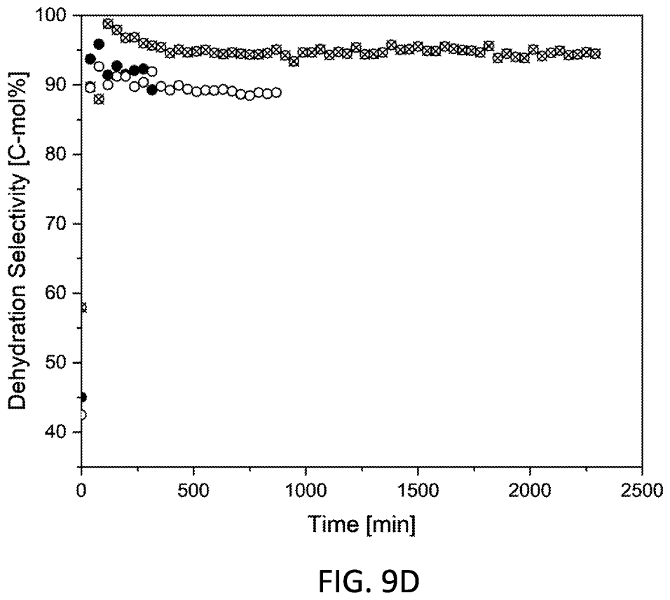

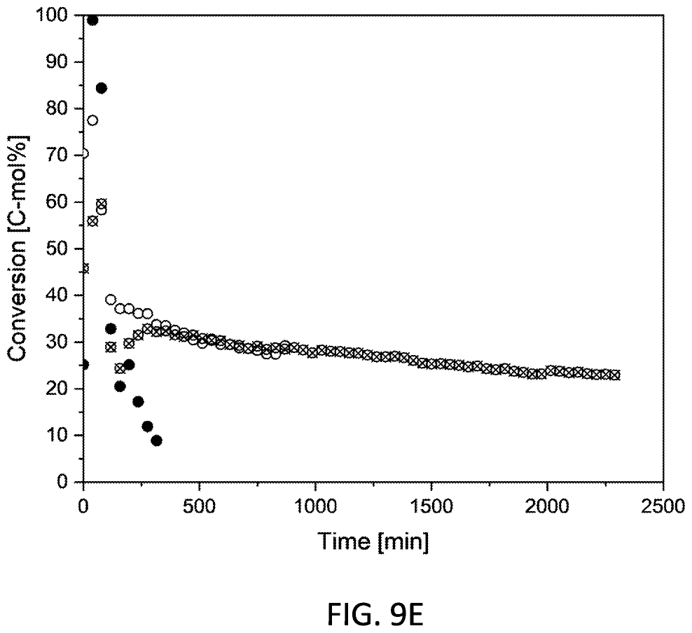

The liquid feed containing methyl lactate and 1,2BPE was introduced to the calcined Na-FAU catalyst bed. A dehydration selectivity of 90 C-mol % was achieved in the first 80 min TOS with conversion above 60 C-mol % (FIG. 9A, filled squares). The comparative modifier, Pyridine, only improves dehydration selectivity to 73 C-mol % (Table 3).

In an alternative mode of introducing the multi-functional modifier pausing the continuous introduction of diamine may provide enough diamine to modulate dehydration selectivity while maintaining a moderate conversion above 10 C-mol %. A separate feed stream with 30 wt % was introduced to a second vaporizer, while the feed stream containing methyl lactate and diamine (here, 1,2BPE) was introduced to the first vaporizer. In this configuration, only the feed stream with methyl lactate and diamine was first introduced to the catalyst bed. At 40 min TOS, the feed stream with methyl lactate and diamine was paused and a valve was switched to allow the feed with only methyl lactate to flow through Na-FAU catalyst bed. The reaction condition here is defined as paused flow condition. A steady dehydration selectivity was observed over 90 C-mol % and steadily increased to over 97 C-mol % after 12 h TOS (FIG. 9A, unfilled squares). A recovery in conversion from 14 C-mol % at 40 min to over 30 C-mol % at 13 h TOS was also observed after no introduction of diamine into the reaction system at 40 min TOS (FIG. 9B, unfilled squares), while under continuous flow condition, conversion rapidly plummeted below 10 C-mol % after 2 h TOS (FIG. 9B, filled squares). The same selectivity was achieved in an independent run with 1,2BPE (FIG. 9C, stars), proving the reproducibility of the over 90 C-mol % dehydration selectivity with 1,2BPE under paused flow condition.

A slightly higher dehydration selectivity of 93 C-mol % under continuous flow condition (FIG. 9C, filled circles) was achieved with 4,4′trimethylenedipyridine (4,4′TMDP), which has one more carbon between the two pyridines than 1,2BPE (Table 5).

TABLE 5
A list of multifunctional components examined in this
invention. Entries A and B are comparative examples.

| | Amine | IUPAC | CAS |
|---|---|---|---|
| Label | Acronym | Name | Number |
| A | No amine | — | — |
| B | Py | pyridine | 110-86-1 |
| C | 2,2′DP | 2,2′-ethylenedipyridine | 4916-40-9 |
| D | 4,4′DP | 4,4′-dipyridyl | 553-56-4 |
| E | 1,2BPE | 1,2-bis(4-pyridyl)ethane | 4916-57-8 |
| F | 2,2′EDP | 2,2′-trimethylenedi- | 16858-01-8 |
| | | pyridine | |
| G | 4,4′TMDP | 4,4′-trimethylenedi- | 16858-01-8 |
| | | pyridine | |
| H | TMPDA | tetramethyl-1,3- | 110-95-2 |
| | | propanediamine | |
| I | TMBDA | tetramethyl-1,4- | 111-51-3 |
| | | butanediamine | |
| J | TMHDA | tetramethyl-1,6- | 111-18-2 |
| | | hexanediamine | |
| K | p-XDA | p-xylylenediamine | 539-48-0 |
| L | m-XDA | m-xylylenediamine | 1477-55-0 |
| M | p-PDA | p-phenylenediamine | 106-50-3 |
| N | p-TMPDA | N,N,N′,N′-tetramethyl- | 100-22-1 |
| | | p-phenylenediamine | |
| O | 1,3CHBMA | 1,3-cyclohexanebis(methylamine | 2579-20-6 |

The conversion also decreased rapidly below 10 C-mol % after 5 h TOS (FIG. 9E, filled circles). A paused flow experiment was also conducted with 4,4′TMDP where the introduction of diamine was paused at 40 min TOS. A steady selectivity over 90 C-mol % was achieved with at least 30 C-mol % conversion over at least 15 h TOS (FIGS. 9D and 9E, unfilled circles) under paused flow condition. Noticeably, dehydration selectivity with 4,4′TMDP was not as high as that with 1,2BPE under paused flow condition. Another paused flow experiment with 4,4′TMDP was conducted where the diamine flow was paused after 120 min TOS instead. A higher dehydration selectivity of 95 C-mol % and a conversion of at least 25 C-mol % (FIGS. 9D and 9E, unfilled squares with cross) was achieved over 36 h TOS.

In addition to gaseous phase deposition in continuous flow or paused flow experiments, multifunctional components comprising diamines can also be introduced via wet impregnation. A certain mass of Diamine was first dissolved in 2 g of methanol according to the specified weight percentage loading of 1, 5, 10, 25, or 40 wt %. 500 mg of dried Na-FAU was then added to the methanol solution. The mixture was stirred at 360 rmp at room temperature for 4 h, after which the mixture was dried in an oven at 70° C. for 24 h. The diamine loadings on Na-FAU were characterized using thermogravimetric analysis (TGA) to match with the intended loadings. The diamine loaded samples were used to dehydration methyl lactate once the temperature of catalyst bed reaches 300° C. with a temperature ramp of 3° C./min from 49° C.

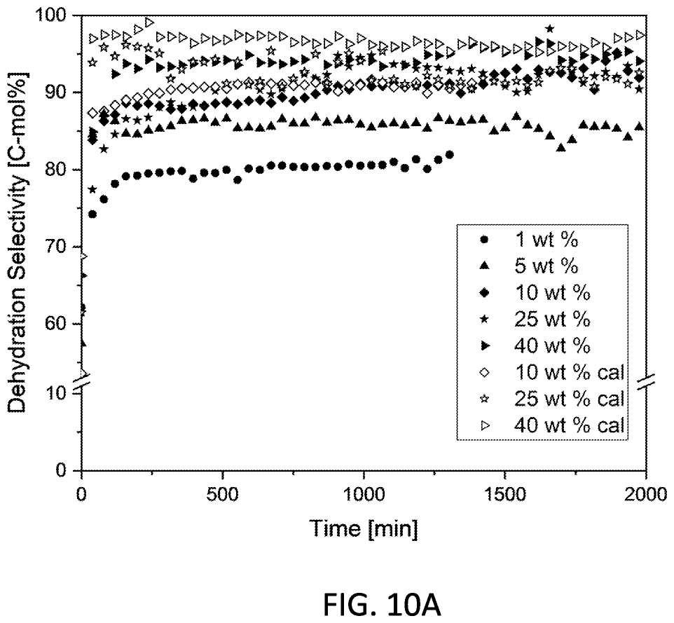

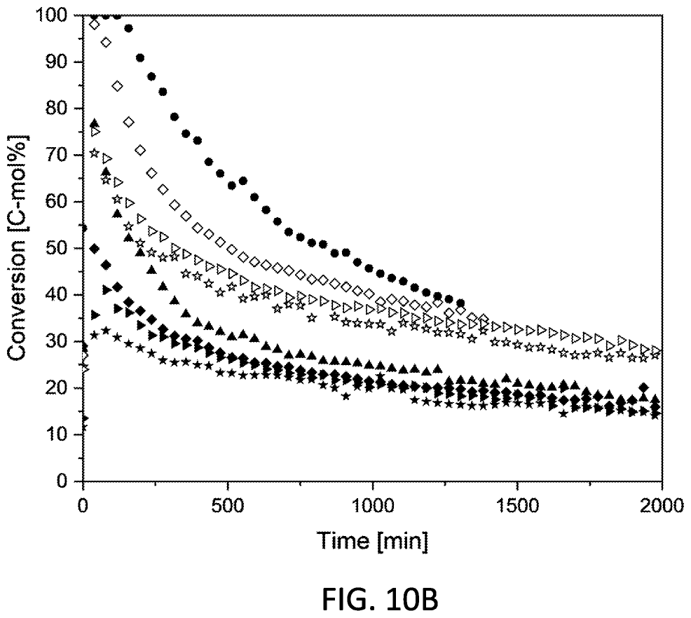

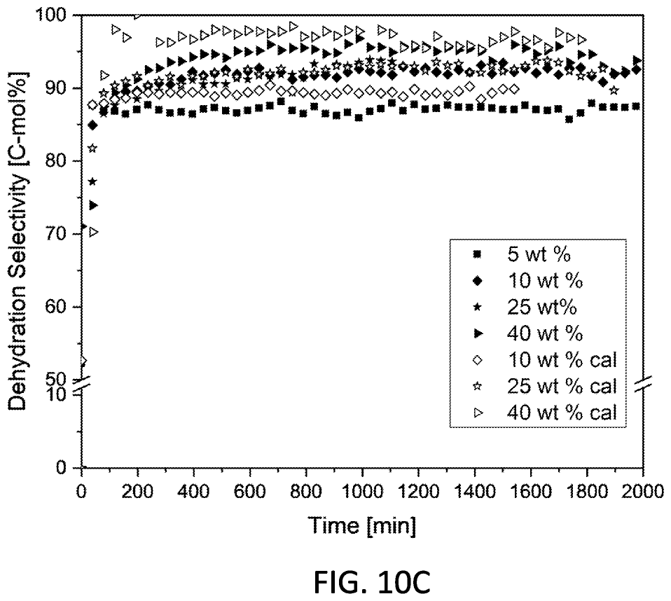

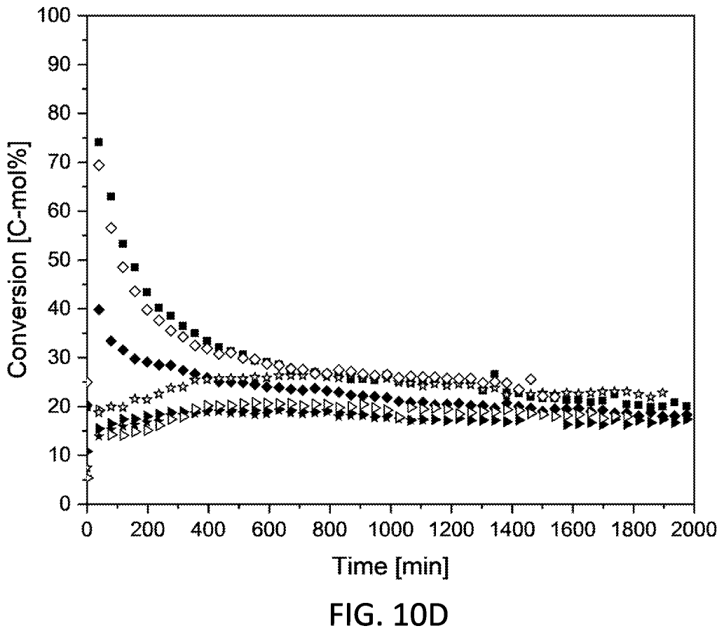

FIGS. 10A-10D show the dehydration selectivity and conversion of methyl lactate over diamine loaded Na-FAU samples with varying amine loadings from 1 to 40 wt %. Data with 4,4′TMDP (4,4′-trimethylenedipyridine) are shown in FIGS. 10A and 10B. Data with 1,2BPE (1,2-bis(4-pyridyl)ethane) are shown in FIGS. 10C and 10D. Data collected with no pre-treatment step are presented in filled symbols and data with pre-treatment step are presented in unfilled symbols. Data collected with a pre-treatment step, denoted as cal, of exposing loaded samples to helium for 500 min before introducing methyl lactate are presented as unfilled symbols.

Dehydration selectivity increases with the increase in multifunctional component loadings from 1 to 40 wt % for both 4,4′TMDP (FIG. 10A, filled circles, triangles, diamonds, stars, and right-pointing triangles) and 1,2BPE (FIG. 10C, filled squares, diamonds, starts, and right-pointing triangles). Under the described condition, the highest dehydration selectivity achieved by both 4,4′TMDP and 1,2BPE was around 95 C-mol % at 40 wt % loading. However, induction periods were observed for the diamine loaded Na-FAU samples of varying loadings, which could be caused by pore blockages due to excess diamines. Such pore blockages can be reduced over time as some of the diamines leave the pores to achieve optimal diamine loading for the high dehydration selectivity.

A pre-treatment step, denoted as cal, exposed loaded samples to helium for 500 min before introducing methyl lactate. The lengths of induction period were significantly reduced with pre-treatment (FIG. 10A, unfilled diamonds, starts, and right-pointing triangles and FIG. 10C, unfilled diamonds, starts, and right-pointing triangles). Dehydration selectivity of 97 C-mol % was achieved with 40 wt % loaded 4,4′TMDP and 1,2 BPE samples with pre-treatment over at least 33 h TOS.

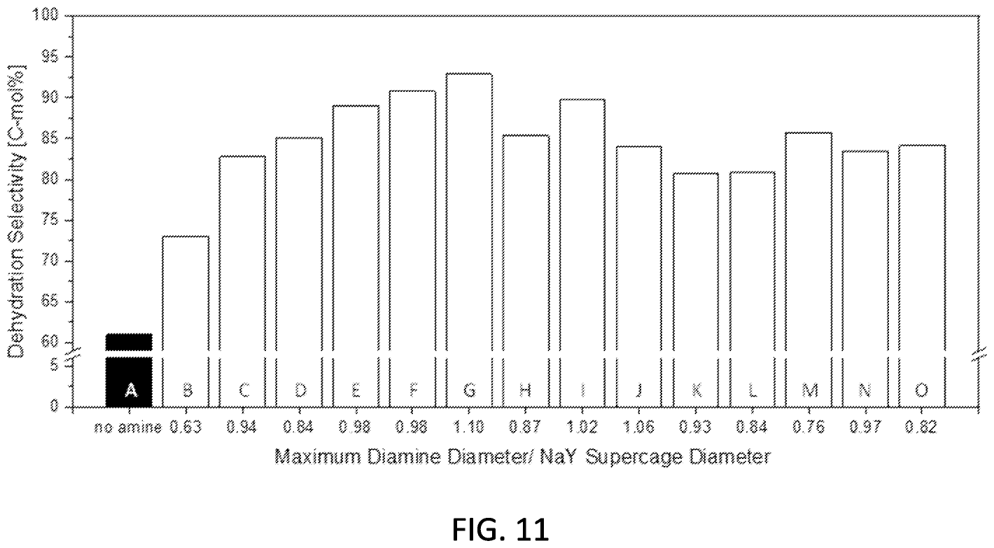

The concept of using multifunctional components with multiple basic functional groups for methyl lactate dehydration selectivity control was examined with diamines that have two amine groups connected by an alkyl chain, an aromatic ring, or an alkyl ring. Under continuous flow conditions, most diamines in FIG. 11 and Table 5 yield methyl lactate dehydration selectivity above 85 C-mol %, with some above 90 C-mol %. However, the increase in dehydration selectivity from 4,4′-dipyridyl, to 1,2BPE, and to 4,4′-TMDP indicates that flexibility between the two amine functional groups may afford additional selectivity control.

The ability of titrant to bind onto multiple acid sites with high flexibility and low Gibbs free energy change may afford higher dehydration selectivity. The maximum size of diamines is calculated using QSAR Toolbox to account for the maximum spread of the diamines and the potential to cover sites that are far apart. A normalized diamine size is calculated by dividing the maximum size by the Na-FAU supercage diameter. As the normalized diamine size (relative to the size of the supercage) approaches 1 or even exceeds 1, the diamine would be able to reach two sites at the opposite ends of the supercage. Another desirable metric quantifies the distance between two amine functional groups; this can be determined by measuring the distance between the two nitrogens on a diamine at the fully relaxed state calculated using VASP. Dehydration selectivity is observed to be positively correlated with the distance between the two nitrogens, indicating that the ability to titrate two sites separated by a larger range of distance may result in higher dehydration selectivity.

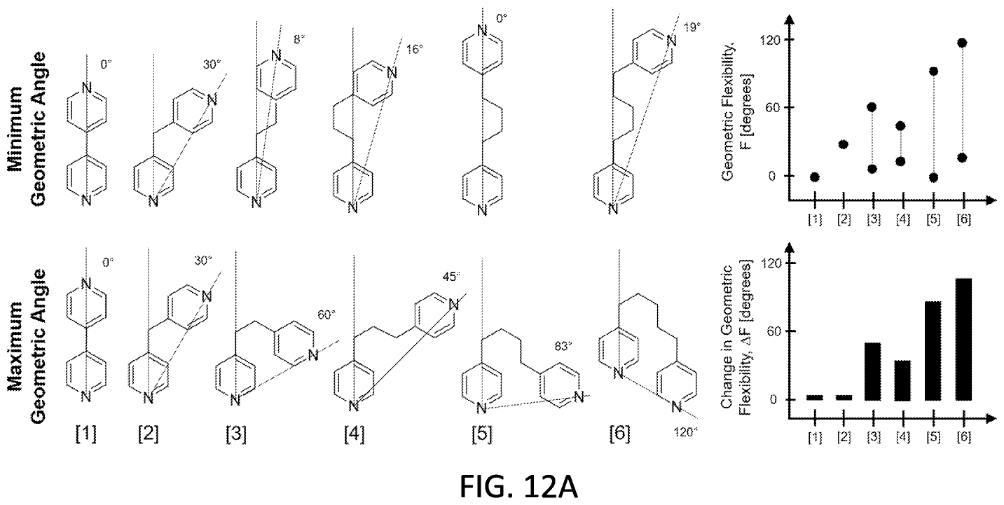

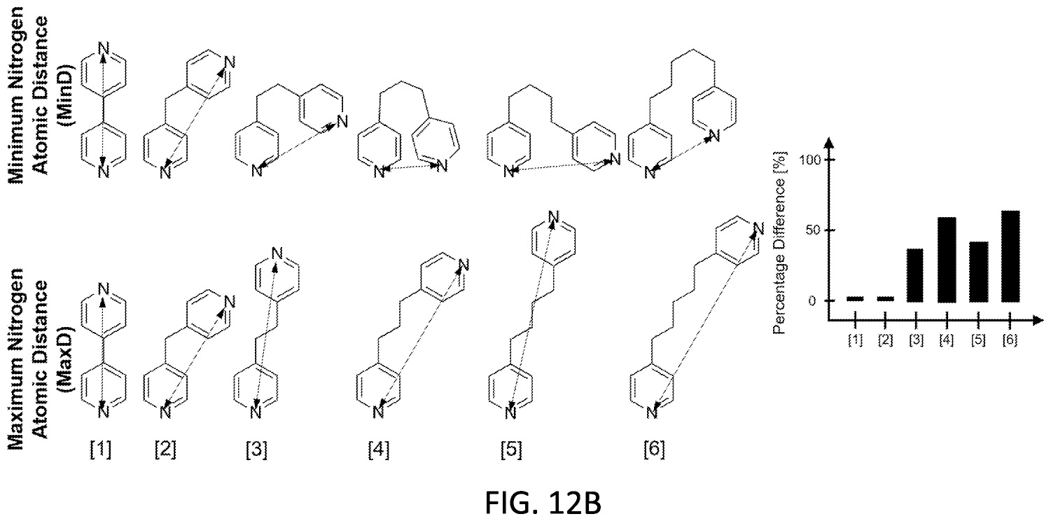

In addition to sites that are far apart, the longer alkyl chain between the two amine groups may also allow the two amines to titrate two adjacent sites due to the increased flexibility of the molecule. Molecular flexibility derives from both the rotational ability of sequential chemical bonds as well as distortion of bonds from their lowest energy conformations. To assess rotational flexibility (a source of molecular flexibility), two descriptors of molecular flexibility are defined:
Geometric Flexibility Angle (GFA)—Using the baseline of the normal axis of one functional site (e.g., amine) for adsorption to the surface, the geometric flexibility angle is the angle or set of angles accessible by the second functional site (e.g., second amine).
Delta Geometric Flexibility (DGF)—The DGF is the extent of angles between the minimum and maximum geometric flexibility angle (GFA).
   Examples of the GFA and DGF are depicted in FIG. 12A for several dipyridines separated by zero to five carbon alkyl chains. Molecule flexibility can also be defined by the difference between the minimum and maximum nitrogen atomic distances (FIG. 12B), where the percentage difference is defined by Equation 9.

$$Percentage Difference = \frac{Maximum Distance - Minimum Distance}{Maximum Distance} \times 100 \% \qquad ( 9 )$$

A flexible molecule should have a DGF of at least 5° or a percentage difference of at least 5%. In an aspect, the GFA is greater than 7° or greater than 100 or greater than 15°. In an aspect, the DGF may be greater than 7° or greater than 100 or greater than 15°. This flexibility plays a role in permitting the reaction modifiers to enhance selectivity to acrylic acid by dehydration of lactic acid; more flexibility may increase acrylic acid production.

The approach of using titrants with multiple basic functional groups was unexpected for methyl lactate dehydration selectivity. The further selectivity enhancement compared with that attained with single amines was not due to higher proton affinity of the diamines. As the distance between N centers increases, the basicity of the second nitrogen becomes higher and closer to that of a single amine. Without being bound by theory, the flexibility due to the longer alkyl chain between two amine groups appears to not only improve coverage on a larger range of sites (both far away and adjacent), but also enhance the binding strength, due to higher basicity, of the second nitrogen once one nitrogen end is anchored on a Bronsted acid site. The flexibility between the two amine groups may also contribute to a lower change in Gibbs free energy for adsorption of one diamine than two single amines on two Bronsted acid sites.

TABLE 6
Calculated proton affinities (PAFF) are summarized below
for multifunctional components comprising diamines shown
in FIGS. 8A and 8B and the corresponding comparative single
amines, pyridine (Py) and trimethylamine (TMA). Diamines
have two PAFF values from protonation of one amine nitrogen
and subsequent protonation of the other nitrogen.

| | PAFF [kJ/mol] | |
|---|---|---|
| | A + H⁺ --> | AH⁺ + H⁺ --> |
| Amine (name, CAS Number) | AH⁺ | AH₂²⁺ |
| Py (pyridine, 110-86-1) | 937 | — |
| 4,4′DP (4,4′-dipyridyl, 553-56-4) | 944 | 664 |
| 1,2BPE (1,2-bis(4-pyridyl)ethane, | 949 | 751 |
| 4916-57-8) | | |
| 4,4′TMDP (4,4′-trimethylenedi- | 953 | 777 |
| pyridine, 16858-01-8) | | |
| TMA (trimethylamine, 75-50-3) | 945 | |
| TMPDA (tetramethyl-1,3- | 964 | 674 |
| propanediamine, 110-95-2) | | |
| TMBDA (tetramethyl-1,4- | 965 | 722 |
| butanediamine, 111-51-3) | | |
| TMHDA (tetramethyl-1,6- | 968 | 781 |
| hexanediamine, 111-18-2) | | |
| p-XDA (p-xylylenediamine, | 940 | 675 |
| 539-48-0) | | |
| m-XDA (m-xylylenediamine, | 937 | 669 |
| 1477-55-0) | | |
| p-PDA (p-phenylenediamine, | 916 | 540 |
| 106-50-3) | | |
| p-TMPDA (N,N,N′,N′-tetramethyl- | 964 | 674 |
| p-phenylenediamine, 100-22-1) | | |
| 1,3CHBMA | 943 | 671 |
| (1,3-cyclohexanebis(methylamine), | | |
| 2579-20-6) | | |

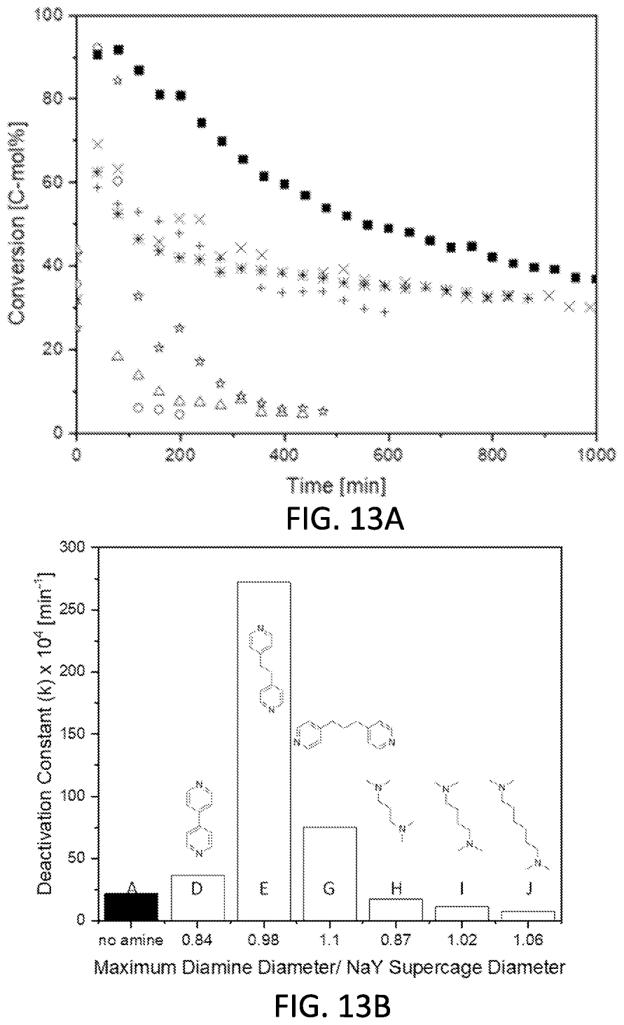

Tetramethyl-1,3-propanediamine (TMPDA), tetramethyl-1,4-butanediamine (TMBDA), and tetramethyl-1,6-hexanediamine (TMHDA) also offer 85 C-mol %, 90 C-mol %, and 84 C-mol % dehydration selectivity respectively (FIG. 11). Moreover, methyl lactate conversion with these dialkylamines are above 30 C-mol % until 10 h TOS, while the conversion with the dipyridines drops rapidly below 10 C-mol after 5 h TOS. Catalysts may be more stable with dialkylamines than dipyridines (FIG. 13A). A first order deactivation model was fitted to the conversion curves in FIG. 13A using Equation 6. The deactivation constants with the dialkylamines are one or two orders of magnitude lower than those with dipyridines (FIG. 13B). Compared with dipyridines, dialkylamines offer high selectivity of 90 C-mol % with higher conversion and slower deactivation under continuous flow condition.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A composition comprising:
a solid acid catalyst comprising a multiplicity of acid sites on the surfaces; and
a multifunctional component coupled to the surfaces of the solid acid catalyst, wherein each multifunctional component comprises at least two functional groups configured to accept a proton from a Bronsted acid site of the multiplicity of acid sites.

2. The composition of claim 1, further comprising a cation comprising hydrogen, ammonium, lithium, sodium, potassium, cesium, magnesium, calcium, strontium, barium, or a combination thereof.

3. The composition of claim 1, wherein the solid acid catalyst comprises a zeolite.

4. (canceled)

5. The composition of claim 3, wherein the zeolite comprises a FAU zeotype.

6. (canceled)

7. The composition of claim 1, wherein the solid acid catalyst comprises one or more of silica-alumina, MCM-41, silica gel, metal organic frameworks, and covalent organic frameworks.

8. (canceled)

9. The composition of claim 1, wherein the solid acid catalyst comprises aluminum.

10. (canceled)

11. (canceled)

12. The composition of claim 1, wherein at least one of the at least two functional groups is an amine functional group.

13. The composition of claim 12, wherein at least two of the at least two functional groups are amine functional groups.

14. The composition of claim 1, wherein the multifunctional component comprises one of the Class I compounds depicted in FIG. 14, wherein each of R₁-R₅ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

15. The composition of claim 1, wherein the multifunctional component comprises one of the Class II and Class III compounds depicted in FIG. 15, wherein each of R₆-R₁₀ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

16. The composition of claim 1, wherein the multifunctional component comprises one of the Class IV compounds depicted in FIG. 16, wherein each R independently represents H, methyl, ethyl, propyl, or isopropyl, and R₁₁ independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

17. The composition of claim 1, wherein the multifunctional component comprises one of the Class V and Class VI compounds depicted in FIG. 17, wherein each R independently represents an alkyl, heteroalkyl, alkene, or heteroalkene group with at least 1 and less than 20 carbon atoms, and wherein each heteroalkyl or heteroalkene group independently comprises one or more of S, Cl, Br, B, F, Si, P, N, and O.

18. The composition of claim 1, wherein the multifunctional component comprises one of the compounds depicted in FIG. 18.

19. The composition of claim 1, wherein, using a baseline of a normal axis of a first one of the at least two functional groups, a geometric flexibility angle of a multifunctional component is the angle or set of angles accessible by a second one of the at least two functional groups, and the geometric flexibility angle is greater than 5 degrees.

20. The composition of claim 19, wherein the multifunctional component comprises a minimum geometric flexibility angle and a maximum geometric flexibility angle, and a difference between the minimum geometric flexibility angle and the maximum geometric flexibility angle is greater than 5 degrees.

21. The composition of claim 1, wherein the at least two functional groups are separated by a spacer comprising an alkyl group, a heteroalkyl group, an alkenyl group, a heteroalkenyl group, a cycloalkyl group, a heterocyclyl group, an aryl group, or a heteroaryl group.

22.-61. (canceled)

62. A composition comprising:
a solid acid comprising a multiplicity of acid sites on the surfaces;
a multifunctional component comprising at least two functional groups, each functional group configured to accept a proton from a Bronsted acid site; and
a reactant comprising lactic acid, a lactic acid ester, a lactic acid salt, or a combination thereof.

63. The composition of claim 62, further comprising a cation comprising hydrogen, ammonium, lithium, sodium, potassium, cesium, magnesium, calcium, strontium, barium, or a combination thereof.

64. The composition of claim 62, wherein the solid acid comprises a zeolite.

65. (canceled)

66. The composition of claim 62, wherein the zeolite comprises a FAU zeotype.

67. (canceled)

68. The composition of claim 62, further comprising acrylic acid, acrylic acid ester, or acrylic acid salt.

69. (canceled)
